(19) United States
(12) Patent Application Publication
(10) Pub. No.: US 2024/0011991 A1
(43) Pub. Date: Jan. 11, 2024

(54) COMPOSITIONS AND METHODS FOR PREDICTING RISK OF MODERATE TO SEVERE COVID-19 DISEASE

(71) Applicant: The Wistar Institute of Anatomy and Biology, Philadelphia, PA (US)

(72) Inventor: Mohamed Abdel-Mohsen, Wynnewood, PA (US)

(21) Appl. No.: 18/252,115

(22) PCT Filed: Nov. 11, 2021
(86) PCT No.: PCT/US2021/058994

§ 371 Date: May 8, 2023

Related U.S. Application Data
(60) Provisional application No. 63/112,547, filed on Nov. 11, 2020.

(51) Int. Cl. G01N 33/569; A61K 45/06; A61K 38/08

(52) U.S. Cl. CPC G01N 33/56983; A61K 45/06; A61K 38/08; G01N 2800/50

(57) ABSTRACT

Compositions, kits and methods for diagnosing and treating an increased risk of moderate to severe COVID-19 disease involves a composition comprising at least one reagent capable of detecting, binding, specifically complexing with, or measuring the level of one of a subject marker in a sample. Optionally, a composition of the invention further comprises a reagent capable of detecting, binding, specifically complexing with, or measuring the expression of a subject biomarker.

### STATEMENT OF GOVERNMENT INTEREST

This invention was made with government support under grant No. DK123733-01S1 and CA010815 awarded by the National Institutes of Health. The government has certain rights in the invention.

### BACKGROUND OF THE INVENTION

Coronavirus Disease 2019 (COVID-19), the disease caused by severe acute respiratory syndrome coronavirus 2 (SARS-CoV-2) infection, can manifest with diverse clinical presentations. While the majority of infected individuals exhibit asymptomatic or mild respiratory tract infection, a significant population, especially those who are older or suffering from pre-existing metabolic-associated diseases, face severe manifestations such as acute respiratory distress syndrome (ARDS), multi-organ failure, and death.¹⁻⁵ A state of hyper-inflammation and hyperactivated immune responses, characterized by an ensuing cytokine storm and increased complement activation, has been associated with COVID-19 severity.^{1,6-10} However, the pathophysiological mechanisms that contribute to these phenomena remain mostly unknown. Understanding these mechanisms is a crucial step in designing rational clinical and therapeutic strategies.

A disruption of the crosstalk between gut microbiota and the lung (gut-lung axis) has been implicated as a driver of severity during respiratory-related diseases, including ARDS.¹¹⁻¹⁴ Systemic inflammation caused by a lung infection or injury can lead to a disruption of the gut barrier integrity and increase the permeability to gut microbes and microbial products. This microbial translocation can exacerbate systemic inflammation and lung injury—resulting in positive feedback.¹¹⁻¹⁴ In addition, SARS-CoV-2 can directly infect gut cells,¹⁵ and viral infections of the gut cause changes in gut structure¹⁶ and breakdown of the epithelial barrier.^{17,18} Such disruption of the gut-lung axis is more likely to occur in older individuals and individuals with metabolic- and/or aging-associated diseases, as these individuals often experience changes in the composition of the gut microbiota (microbial dysbiosis),^{19,20} which facilitate a higher susceptibility to falling into the vicious cycle between microbial translocation and systemic inflammation.²¹⁻²⁴

Even as microbial translocation impacts systemic inflammation directly, it may also impact it indirectly by modulating circulating levels of gut- and gut microbiota-associated products such as metabolites and lipids. Plasma metabolites and lipids can reflect the functional status of the gut and the metabolic activity of its microbiota.²⁵⁻²⁸ They also are biologically active molecules in their own right, regulating several immunological functions, including inflammatory responses.^{29,30} A third class of microbial products that can translocate from the gut is glycan-degrading enzymes. Glycans on circulating glycoproteins and antibodies (IgGs and IgAs) are essential for regulating several immunological responses, including complement activation.³¹ The glycan-degrading enzymes are released by several members of the gut microbiome and their translocation can alter the circulating glycome, leading to higher inflammation and complement activation.³² Indeed, altered glycosylation of plasma glycoproteins (including immunoglobulin G, IgG) has been associated with the onset and progression of inflammatory bowel disease (IBD).³²⁻³⁷ Furthermore, modulation of the gut microbiota via fecal microbiota transplantation affects IgG and serum glycosylation.³⁸

There remains a need in the art for more accurate and sensitive diagnostic assays for predicting increased risk of moderate or severe COVID-19.

### SUMMARY OF THE INVENTION

In one aspect, a method for detecting an increased risk of moderate or severe illness in a subject is provided. In one embodiment, the method includes detecting the level of one or more subject biomarker of intestinal barrier integrity or inflammation in a sample from a subject having, or suspected of having, an illness associated with a coronavirus infection; comparing the level of the one or more subject biomarkers to a control level; and diagnosing the subject with an increased risk of moderate or severe respiratory illness when an increase in the level of one or more subject biomarkers is detected as compared to a control. In one embodiment, the method includes treating the subject for moderate or severe respiratory illness when an increased risk is detected.

In another embodiment, the method includes detecting the levels of metabolites in the plasma of a subject having, or suspected of having an illness associated with a coronavirus infection; comparing the levels of the metabolites to control levels; and diagnosing the subject with a higher risk of moderate or severe respiratory illness when a significant change is detected in 10 or more metabolites in the subject's plasma as compared to a control. In one embodiment, the method includes treating the subject for moderate or severe respiratory illness.

In another embodiment, the method includes detecting the level of citrulline in the plasma of a subject having, or suspected of having an illness associated with a coronavirus infection; comparing the levels of citrulline to a control level; and diagnosing the subject with a higher risk of moderate or severe respiratory illness when a significant decrease is detected in the citrulline level in the subject's plasma as compared to a control. In one embodiment, the method includes treating the subject for moderate or severe respiratory illness.

In another embodiment, the method includes detecting the level of succinic acid in the plasma of a subject having, or suspected of having an illness associated with a coronavirus infection; comparing the levels of succinic acid to a control level; and diagnosing the subject with a higher risk of moderate or severe respiratory illness when a significant increase is detected in the succinic acid level in the subject's plasma as compared to a control. In one embodiment, the method includes treating the subject for moderate or severe respiratory illness.

In another embodiment, the method includes detecting the ratio of kynurenine/tryptophan [Kyn/Trp] in the plasma of a subject having, or suspected of having an illness associated with a coronavirus infection; comparing the ratio of Kyn/Trp to a control level; and diagnosing the subject with a higher risk of moderate or severe respiratory illness when a significant increase is detected in the Kyn/Trp ratio in the subject's plasma as compared to a control. In one embodiment, the method includes treating the subject for moderate or severe respiratory illness.

In some embodiments, the method further includes measuring the level of IL-6, where an increase in IL-6 is further indicative of increased risk of moderate or severe respiratory illness.

In certain embodiments, the treatment comprises a treatment to repair or improve gut barrier integrity. In another embodiment, the treatment includes oxygen therapy, remdesivir, dexamethasone (or other corticosteroid), treatment to reduce gut permeability, or dietary change. In another embodiment, the treatment includes decreasing the levels of zonulin in the subject.

In another aspect, a method of treating COVID-19 disease in a subject includes administering a zonulin receptor antagonist.

In another aspect, a method of treating COVID-19 disease in a subject includes increasing the level of citrulline level in the subject thru diet or pharmaceutical intervention.

In another aspect, a method of treating COVID-19 disease in a subject includes inhibiting or reducing the level of one or more galectins in the subject. In one embodiment, the galectin is GAL-3 or GAL-9.

In another aspect, a method of detecting an increased risk of developing, or diagnosing a patient with, Long-COVID is provided. In one embodiment, the method includes detecting the levels of biomarkers in the plasma of a subject having, or suspected of having an illness associated with a coronavirus infection; comparing the levels of the biomarkers to control levels; and diagnosing the subject with a higher risk of moderate or severe respiratory illness when a significant change is detected in β-glucan in the subject's plasma as compared to a control. In one embodiment, the method includes treating the subject for Long-COVID.

In another aspect, a method for detecting an increased risk of death in a subject is provided. The method includes detecting the level of one or more subject biomarkers of intestinal barrier integrity or inflammation in a sample from a subject having, or suspected of having, an illness associated with a coronavirus infection; comparing the level of the one or more subject biomarkers to a control level; and diagnosing the subject with an increased risk of death when an increase in the level of one or more subject biomarkers is detected as compared to a control. In one embodiment, the method includes treating the subject for moderate or severe respiratory illness when an increased risk is detected.

In one embodiment, of the methods described herein, the subject has COVID-19 disease.

In one aspect, a composition is provided which allows for the detection, or measurement of, one or more subject biomarker in a biological sample. In one embodiment, the composition includes at least one reagent capable of detecting, binding, specifically complexing with, or measuring the level of a biomarker in a sample selected from those in Table 1. In one embodiment, the composition includes multiple reagents, each capable of detecting, binding, specifically complexing with, or measuring the level of one of a biomarker selected from those of Table 1.

In another aspect, a kit is provided. In one embodiment, the kit includes reagents capable of detecting, binding, specifically complexing with or measuring the level of one or more of the biomarkers from Table 1.

Other aspects and advantages of the invention are described further in the following detailed description of the preferred embodiments thereof.

### BRIEF DESCRIPTION OF THE DRAWINGS

FIG. 1A-FIG. 1G show COVID-19 is associated with an increase in markers of tight junction permeability and microbial translocation. (FIG. 1A) An overview of the study design; figures in black indicate deceased; moderate and severe patients were hospitalized; severe indicates patients in the intensive care unit. (FIG. 1B) Levels of plasma zonulin, a marker of tight junction permeability, are higher during moderate and severe COVID-19 compared to mild COVID-19 or controls. Kruskal-Wallis test was used for statistical analysis. False discovery rate (FDR) was calculated using the Benjamini-Hochberg method. Symbols in black indicate deceased. (FIG. 1C) Zonulin levels are higher in hospitalized COVID patients (n=40) who eventually died from COVID-19 (n=8) compared to survivors (n=32). Nominal P-value was calculated using the Mann-Whitney U test. (FIG. 1D-FIG. 1G) Levels of markers of translocation and inflammation, LBP (FIG. 1D), β-Glucan (FIG. 1E), sCD14 (FIG. 1F), and MPO (FIG. 1G) are higher during severe COVID-19 compared to mild COVID-19 or controls. Kruskal-Wallis test was used for statistical analysis. FDR was calculated using Benjamini-Hochberg method.

FIG. 2A-FIG. 2H show markers of tight junction permeability and microbial translocation are linked to systemic inflammation and immune dysfunction. (FIG. 2A-FIG. 2C) levels of representative variables, galectin-3 (Gal-3) (FIG. 2A) and galectin-9 (Gal-9) (FIG. 2B), were higher during severe COVID-19 compared to mild COVID-19 or controls, with levels of Gal-9 higher among deceased hospitalized patients compared to survivors (FIG. 2C). (FIG. 2D-FIG. 2F) Levels of C3a (FIG. 2F) and GDF-15 (FIG. 2E) were higher during severe COVID-19 compared to mild COVID-19 or controls, with levels of GDF-15 higher among deceased hospitalized patients compared to survivors (FIG. 2F). Kruskal-Wallis and Mann-Whitney tests were used for statistical analysis. FDR was calculated using Benjamini-Hochberg method. (FIG. 2G-FIG. 2H) Examples of correlations between LBP and IL-6 (FIG. 2G) or β-Glucan and IL-6 (FIG. 2H). Spearman's rank correlation tests were used for statistical analysis.

FIG. 3A-FIG. 3H show severe COVID-19 is associated with metabolic dysregulation in a manner linked to gut dysfunction. (FIG. 3A) Principal component analysis (PCA) of the 278 metabolites identified in the plasma of the study cohort. Each symbol represents a study participant. (FIG. 3B) Ingenuity Pathway Analysis (IPA) of the plasma metabolites modulated between the disease states with FDR<0.05. The graph shows the top 10 dysregulated metabolic pathways with FDR<0.05. Percentages beside each pathway represent the ratio of dysregulated metabolites among the total number of metabolites assigned to this particular pathway in IPA. (FIG. 3C-FIG. 3E) As representative examples, levels of Citrulline are lower (FIG. 3C), levels of succinic acid are higher (FIG. 3D), and the ratio between kynurenine/tryptophan [Kyn/Trp] is higher (FIG. 3E) during severe COVID-19 compared to mild COVID-19 or controls. Kruskal-Wallis test was used for statistical analysis. FDR was calculated using Benjamini-Hochberg method. (FIG. 3F-FIG. 3G) For key metabolites in the tryptophan catabolism pathway, levels of tryptophan are lower (FIG. 3F), and levels of kynurenic acid are higher (FIG. 3G) in deceased COVID-19 hospitalized patients compared to survivors. Nominal P-value was calculated using the Mann-Whitney U test.

FIG. 4A-FIG. 4C show metabolic markers of intestinal dysfunction are linked to microbial translocation and systemic inflammation. Examples of the correlations between citrulline and IL-6 (FIG. 4A), succinic acid and IL-6 (FIG. 4B), or [Kyn/Trp] ratio and IL-6 (FIG. 4C). Spearman's rank correlation tests were used for statistical analysis. FDR was calculated using Benjamini-Hochberg method.

FIG. 5A-FIG. 5B show severe COVID-19 is associated with disrupted lipid metabolism. (FIG. 5A) Principal component analysis (PCA) of 2015 lipids identified in the plasma of the study cohort. (FIG. 5B) Lipid pathway analysis of the plasma lipids modulated between the disease states with FDR<0.05 was performed using LIPEA (Lipid Pathway Enrichment Analysis; https://lipea.biotec.tudresden.de/home). The graph includes all dysregulated pathways with FDR<0.05. Percentages beside each pathway represent the ratio of dysregulated lipids among the total number of lipids assigned to this particular pathway by LIPEA.

FIG. 6A-FIG. 6D show severe COVID-19 is associated with plasma glycomic dysregulations. (FIG. 6A-FIG. 6B) Levels of terminal digalactosylated N-glycans in IgG (FIG. 6A) or total plasma glycoproteins (FIG. 6B) are lower during severe COVID-19 compared to mild COVID-19 or controls. Kruskal-Wallis test. FDR was calculated using Benjamini-Hochberg method. (FIG. 6C-FIG. 6D) Correlation heat-maps depicting the correlations between galactosylated N-glycans (rows) and markers of tight junction permeability and microbial translocation (FIG. 6C) or markers of inflammation and immune dysfunction (FIG. 6D). SC rho=coefficient of correlation with COVID-19 severity. Red-colored correlations=positive correlations with FDR<0.05, blue-colored correlations=negative correlations with FDR<0.05, and gray-colored correlations=non-significant. Spearman's rank correlation tests were used for statistical analysis. FDR was calculated using Benjamin-Hochberg method.

FIG. 7A-FIG. 7C show logistic models using markers of tight junction permeability and microbial translocation strongly distinguish hospitalized from non-hospitalized individuals. (FIG. 7A) The machine learning algorithm, Lasso (least absolute shrinkage and selection operator) regularization, selected three markers (zonulin, LBP, and sCD14) that, when combined, can distinguish hospitalized (n=40; severe and moderate groups combined) from non-hospitalized (n=40; mild and control groups combined) individuals. The receiver operator characteristic (ROC) curve showing an area under the curve (AUC) of 99.23% from the multivariable logistic regression model with the three variables combined. (FIG. 7B) Coefficients from the multivariable logistic model were used to estimate a hospitalization risk score for each individual and then tested for the ability of these scores to accurately classify hospitalized (n=40) from non-hospitalized (n=39; one sample did not have a complete dataset) individuals at an optimal cut-point. The model correctly classified hospitalized (97.5% sensitivity) and non-hospitalized (94.9% specificity), with an overall accuracy of 96.2%. Squares represent individuals the model failed to identify correctly. (FIG. 7C) Logistic regression model using the L-kynurenine/L-tryptophan [Kyn/Trp] ratio, a marker of gut microbiome dysbiosis, is able to distinguish hospitalized from non-hospitalized individuals. ROC curve showing the area under the curve (AUC) is 91.3%.

FIG. 8 shows a table with levels of the 35 out of 50 gut- and gut microbiota-associated plasma metabolites that are disrupted during COVID-19. Red indicates upregulation, blue indicates downregulation; color intensity indicates larger difference. Green indicates FDR<0.05; color intensity indicates lower FDR.

FIG. 9 shows a table with demographic and clinical characteristics of the study cohort.

FIG. 10 shows a table with a list of plasma markers measured in this study.

FIG. 11 shows a table with the top 50 metabolic pathways disrupted by severe COVID-19.

FIG. 12 shows a table with a list of the gut-associated and gut microbiota-associated metabolites detected in our study using untargeted LC-MS/MS (50 of the 278 metabolites identified in plasma).

FIG. 13 shows a table of the 24 lipid classes to which the two thousand fifteen lipids identified in this study were assigned.

FIG. 14 shows a table with the structures and names of N-glycans identified in plasma by capillary electrophoresis. These glycan structures can be grouped into 15 groups: bisecting GlcNAc (B group), sialic acid (non-siaylated (S0), mono-sialylated (S1), di-sialylated (S2), tri-sialylatedand (S3), tetra-sialylated (S4), and total sialylated (ST)), galactose (agalactosylated (G0), mono-galactosylated (G1), di-galactosylated (G2), and total galactosylated (GT)), core fucose (FC group), branched fucose (FB group), high branched (HB group), and low branch (LB group).

FIG. 15 shows a table with the structures and names of N-glycans identified in IgG by capillary electrophoresis. These glycan structures were grouped into 9 groups, depending on the presence or absence of four key monosaccharides: bisecting GlcNAc (B group), sialic acid (mon-sialylated (S1), di-sialylated (S2), and total siaylated (ST)), terminal galactose (agalactosylated (G0), mono-galactosylated (G1), di-galactosylated (G2), and total galactose (GT)), and fucose (F group).

FIG. 16 shows a table with lectins used in the 45-plex lectin microarray and their glycan-binding specificity.

FIG. 17A-C shows a validation of key measurements in an independent cohort. Levels of plasma (FIG. 17A) zonulin, (FIG. 17B) LBP, and (FIG. 17C) sCD14 are higher during moderate and severe COVID-19 compared to mild COVID-19 or controls in an independent validation cohort. Kruskal-Wallis test was used for statistical analysis.

FIG. 18A-18C are three graphs demonstrating that long-COVID is associated with an increase in markers of fungal translocation. (FIG. 18A) Plasma levels of β-Glucan (a marker of fungal translocation) during acute COVID-19 (mild, moderate, or severe symptoms) compared to SARS-CoV-2 negative controls (n=20 per group). Kruskal-Wallis test was used for statistical analysis. (FIG. 18B) Plasma levels of β-Glucan measured at four months after COVID-19 (negative PCR test) in 56 individuals who are not suffering from 1 or more persistent symptoms (non-Long-COVID) and 61 individuals suffering from persistent symptoms (Long-COVID). Mann-Whitney U test. (FIG. 18C) The 61 Long-COVID patients from panel b were divided into 40 individuals suffering from 1-7 symptoms or 21 individuals suffering from >8 symptoms. Levels of β-Glucan were compared between these two groups and non-Long-COVID controls. Kruskal-Wallis test was used for statistical analysis. ***=P<0.001, *=P<0.05.

### DETAILED DESCRIPTION OF THE INVENTION

The present invention answers the need in the art by providing novel compositions and methods for predicting the increased risk of moderate to severe illness associated with coronavirus infection, as further described herein. The compositions and methods described herein, are suitable for use with both symptomatic and asymptomatic subjects.

It was hypothesized that a vicious cycle between SARS-CoV-2 infection, systemic inflammation, disrupted intestinal barrier integrity, and microbial translocation contributes to COVID-19 severity. To test this hypothesis, the inventors applied a multi-omic systems biology approach to analyze plasma samples from 60 COVID-19 patients with varying disease severity and 20 age-controlled (most were 50 to 65 years old) and gender-matched (˜50% female) SARS-CoV-2 negative controls. The inventors investigated the potential links between gut barrier integrity, microbial translocation, systemic inflammation, and COVID-19 severity. The data indicate that severe COVID-19 is associated with a dramatic increase in tight junction permeability and translocation of bacterial and fungal products into the blood. This disrupted intestinal barrier integrity and microbial translocation correlates strongly with increased systemic inflammation, increased immune activation, decreased intestinal function, disrupted plasma metabolome and glycome, and higher mortality rate.

Coronavirus Disease 2019 (COVID-19) refers to the disease caused by severe acute respiratory syndrome coronavirus 2 (SARS-CoV-2) infection. As used herein, the term “asymptomatic infection” or “presymptomatic infection” and the like, refers to individuals who test positive for SARS-CoV-2 using a virologic test (i.e., a nucleic acid amplification test or an antigen test), but who have no symptoms that are consistent with COVID-19.

As used herein, the term “mild infection” or “mild illness” and the like, refers to individuals who have any of the various signs and symptoms of COVID-19 (e.g., fever, cough, sore throat, malaise, headache, muscle pain, nausea, vomiting, diarrhea, loss of taste and smell) but who do not have shortness of breath, dyspnea, or abnormal chest imaging.

As used herein, in one embodiment, the term “moderate infection” or “moderate illness”, and the like, refers to a respiratory illness requiring hospitalization, including inpatient hospitalization. In one embodiment, the term moderate infection refers to illness which requires inpatient hospitalization on the “regular” hospital wards, i.e., not the Intensive Care Unit. In one embodiment, moderate illness includes individuals who show evidence of lower respiratory disease during clinical assessment or imaging and who have saturation of oxygen (SpO2)>94% on room air at sea level.

As use herein, in one embodiment, the term “severe infection” or “severe symptoms” and the like, refers to a respiratory illness requiring hospitalization, including inpatient hospitalization, at a level higher than the “regular” hospital ward. For example, in one embodiment, the term severe infection refers to illness which requires inpatient hospitalization on the Intensive Care Unit (ICU) hospital wards, i.e., not the Intensive Care Unit. In another embodiment, severe infection refers to illness requiring intubation. In another embodiment, severe infection refers to individuals who have SpO2<94% on room air at sea level, a ratio of arterial partial pressure of oxygen to fraction of inspired oxygen (PaO2/FiO2)<300 mmHg, respiratory frequency >30 breaths per minute, or lung infiltrates >50%. In another embodiment, severe infection refers to individuals who have respiratory failure, septic shock, and/or multiple organ dysfunction.

The term “Long-COVID” as used herein, refers to symptoms that continue past the initial acute phase of illness. The condition occurs in individuals with a history of probable or confirmed SARS-CoV-2 infection, usually 3 months from the onset of COVID-19 with symptoms that last for at least 2 months and cannot be explained by an alternative diagnosis. Common symptoms include fatigue, shortness of breath, and cognitive dysfunction, among others, and generally have an impact on everyday functioning. Symptoms may be of new onset following initial recovery from an acute COVID-19 episode or persist from the initial illness. Symptoms may also fluctuate or relapse over time.

“Increased likelihood” or “increased risk” of developing moderate or severe infection, as used herein, means an increase in the risk or probability that the subject will develop moderate or severe infection, as compared to a predetermined control or baseline level. In one embodiment, increased likelihood means a 0.5 to 1 fold increase over the control or baseline level. In another embodiment, increased likelihood means a 1.0-1.5 fold increase over the control or baseline level. In one embodiment, increased likelihood means a 1.5 to 2 fold increase over the control or baseline level. In another embodiment, increased likelihood means a 2-3 fold increase over the control or baseline level. In another embodiment, increased likelihood means a 3-4 fold increase over the control or baseline level. In another embodiment, increased likelihood means a 4-5 fold increase over the control or baseline level. In another embodiment, increased likelihood means a 5-6 fold increase over the control or baseline level. In another embodiment, increased likelihood means a 6-7 fold increase over the control or baseline level. In another embodiment, increased likelihood means a 7-8 fold increase over the control or baseline level. In another embodiment, increased likelihood means an 8-9 fold increase over the control or baseline level. In another embodiment, increased likelihood means a 9-10 fold increase over the control or baseline level. In another embodiment, increased likelihood means a 10 fold or greater increase over the control or baseline level. Each of the numbers described above includes the endpoints and any fractions or integers therebetween. The baseline risk of moderate or severe infection may vary based on the subject population (e.g., race, socioeconomic status, smoking status, geographical location, etc.). In another embodiment, increased risk means a 10% greater risk over the control or baseline level. In another embodiment, increased risk means a 30% greater risk over the control or baseline level. In another embodiment, increased risk means a 30% greater risk over the control or baseline level. In another embodiment, increased risk means a 40% greater risk over the control or baseline level. In another embodiment, increased risk means a 50% greater risk over the control or baseline level. In another embodiment, increased risk means a 60% greater risk over the control or baseline level. In another embodiment, increased risk means a 70% greater risk over the control or baseline level. In another embodiment, increased risk means an 80% greater risk over the control or baseline level. In another embodiment, increased risk means a 90% greater risk over the control or baseline level. In another embodiment, increased risk means a 100% or greater risk over the control or baseline level.

“Biological sample” or “sample” as used herein means any biological fluid or tissue that contains the subject biomarkers. In one embodiment, the biological sample is plasma. In one embodiment, the biological sample is serum. Other useful biological samples include, without limitation, whole blood, plasma, gut cells, stool, urine, or saliva. In some examples, “blood” may refer to any blood component used as a sample such as whole blood, plasma or serum. Such samples may further be diluted with saline, buffer or a physiologically acceptable diluent. Alternatively, such samples are concentrated by conventional means.

By the terms “patient” or “subject” as used herein is meant a mammalian animal, including a human, a veterinary or farm animal, a domestic animal or pet, and animals normally used for clinical research, including non-human primates, dogs and mice. More specifically, the subject of these methods is a human. In one embodiment, the subject has, or is suspected of having, a respiratory illness associated with a coronavirus infection. In another embodiment, the coronavirus infection is caused by SARS-COV-2, i.e., COVID-19 disease (also referred to as “COVID-19”). In one aspect of the methods described herein, the subject undergoing the diagnostic or therapeutic method is asymptomatic for respiratory infection or COVID-19. In another aspect, the subject undergoing the diagnostic or therapeutic methods described herein shows clinical indicators, or history, of respiratory infection or COVID-19. In another embodiment, the subject has tested positive for SARS-COV-2. In another embodiment, the subject has COVID-19.

“Clinical indicators of respiratory infection or COVID-19” as used herein, include, but are not limited to, fever, chills, cough, shortness of breath or difficulty breathing, fatigue, muscle or body aches, headache, new loss of taste or smell, sore throat, congestion or runny nose, nausea or vomiting, or diarrhea.

“Healthy subjects” or “healthy control” as used herein refer to a subject or population of multiple subjects that did not test positive for and/or develop symptoms of respiratory infection or COVID-19. In one embodiment, healthy subjects may be a subject or population of multiple subjects that tested positive for SARS-COV-2, but did not develop COVID-19 disease. In another embodiment, the healthy control is an artificial standard, such as that based on collected data from healthy subjects. Such artificial standard may be a standard such as that provided with a kit.

As used herein, the “subject biomarker” refers to one or more of the biomarkers described herein, and contained in Table 1, below.

TABLE 1
Subject Biomarkers

zonulin
β-glucan
LPS binding protein (LBP)
regenerating islet-derived protein 3 alpha (REG3α)
sCD14
myeloperoxidase (MPO)
soluble CD163
IL-6
IL-1β
CRP
d-dimer
galectin-3
galectin-9
C3a
GDF-15
IL-10
MCP-2
IL-15
MIP-1 α
IL-22
TNF-α
IFN-γ
Fractalkine
IP-10
IL-21

In one embodiment, the “subject biomarker” refers to zonulin. In one embodiment, the “subject biomarker” refers to β-glucan. In another embodiment, the “subject biomarker” refers to citrulline. In another embodiment, the “subject biomarker” refers to one or more of zonulin, LPS binding protein, β-glucan, and regenerating islet-derived protein 3 alpha (REG3a). In another embodiment, the “subject biomarker” refers to one or more of sCD14, myeloperoxidase (MPO), soluble CD163, IL-6, IL-1β, CRP, d-dimer, galectin-3, galectin-9, C3a, and GDF-15. In another embodiment, the “subject biomarker” refers to one or more of CRP, IL-1, GDF-15, d-dimer, MPO, IP-10, IL-10, GAL-9, MCP-2, IL-15, MIP-1a, GAL-3, C3a, IL-1B, IL-22, TNF-α, IL-21, and fractalkine. In another embodiment, the “subject biomarker” refers to one or more of CRP, IL-1, GDF-15, d-dimer, MPO, IP-10, IL-10, GAL-9, MCP-2, IL-15, MIP-1a, GAL-3, C3a, IL-1B, IL-22, TNF-α, IL-21, fractalkine, and IFN-γ. In another embodiment, the “subject biomarker” refers to one or more of CRP, IL-1, GDF-15, d-dimer, MPO, IP-10, IL-10, GAL-9, MCP-2, IL-15, MIP-1a, GAL-3, C3a, IL-1B, IL-22, TNF-α, and IFN-γ. In another embodiment, the “subject biomarker” refers to one or more of CRP, IL-1, GDF-15, d-dimer, MPO, IP-IL-10, GAL-9, MCP-2, MIP-1a, GAL-3, C3a, IL-1B, IL-22, and TNF-α. In another embodiment, the “subject biomarker” refers to zonulin, LBP and sCD14. In another embodiment, the “subject biomarker” refers to zonulin and β-glucan. Other combinations of the biomarkers described herein are contemplated by the invention. Any of isoforms of the subject biomarkers may be measured.

As used herein, the term “control” refers to a numerical level, average, mean or average range of the expression of a biomarker in a defined population. The predetermined control level is preferably provided by using the same assay technique as is used for determination of presence or level of the subject biomarker. For example, the control may comprise a single healthy mammalian subject. In another embodiment, the control comprises a single mammalian subject who did not develop symptoms of respiratory infection and/or COVID-19. In another embodiment, the control comprises a population of multiple healthy mammalian subjects or multiple healthy mammalian subjects who did not symptoms of respiratory infection and/or COVID-19.

Provided herein are compositions and methods useful for predicting an increased risk of moderate or severe respiratory illness associated with coronavirus infection utilizing the presence or level of one or more subject biomarkers, as further described herein. In summary, the data here strongly suggest for the first time: (1) severe COVID-19 is associated with disrupted intestinal barrier integrity, higher microbial translocation, and gut dysfunction; (2) severe COVID-19 is associated with a dramatic shift in levels of several biologically active molecules, which likely contribute to disease severity by inducing inflammation. This study sheds light on the potentially critical role of a previously unappreciated factor, disruption of intestinal barrier integrity, in the pathophysiology of severe COVID-19.

A significant strength of our multi-omics approach is its ability to uncover connections between severe COVID-19 and biomolecules of different classes. The carbohydrate structures (glycans) attached to circulating proteins, including antibodies, and their receptors (lectins) are increasingly being appreciated for their essential roles in a variety of immune functions. Among the glycobiological molecules regulated by severe COVID-19 are galectins (increasing) and galactosylated glycans on circulating glycoproteins (decreasing). Both point to a glycomic contribution to the severity of COVID-19. First, galectins (secreted, GalNAc-binding proteins) have emerged as significant modulators of cytokine expression by immune cells during several diseases, including viral infections.⁹³⁻⁹⁵ Importantly, small molecule inhibitors for galectins, especially for Gal-3, can reduce inflammation and cytokine release.^{96,97} Therefore, galectins represent potential therapeutic targets to reduce cytokine storm during COVID-19.^{98,99} Second, galactosylated glycans on circulating antibodies link Dectin-1 on phagocytes to FcγRIIB on myeloid cells to prevent the inflammation mediated by complement activation.³¹ Loss of galactose, as observed during COVID-19, decreases the opportunity to activate this important anti-complement activation checkpoint, thereby promoting inflammation. As using highly-galactosylated immune complexes can prevent inflammation mediated by complement activation,³¹ these and similar glycomic approaches may represent another therapeutic opportunity to reduce inflammation during COVID-19.

Provided herein, in another aspect, are compositions and methods useful for predicting an increased risk of developing Post-Acute COVID-19 Syndrome (Long-COVID) utilizing the presence or level of one or more subject biomarkers, as further described herein. In summary, the data here strongly suggest for the first time that Long-COVID is associated with an increase in markers of fungal translocation

A. Subject Biomarkers

In part, the compositions and methods described herein relate to the presence of certain biomarkers in the serum of COVID-19 patients that correlate with incidence of moderate to severe COVID-19 disease. In other embodiments, the compositions and methods described herein relate to the presence of certain biomarkers in the serum of COVID-19 patients that correlate with the incidence of Long-COVID. In one embodiment, the composition or method includes reagents capable of checking for the presence of, or measuring the level of, a “subject biomarker” selected from those of Table 1.

In one aspect, a composition is provided which allows for the detection, or measurement of, one or more subject biomarker in a biological sample. In one embodiment, the composition includes at least one reagent capable of detecting, binding, specifically complexing with, or measuring the level of a biomarker in a sample selected from those in Table 1. In one embodiment, the composition includes multiple reagents, each capable of detecting, binding, specifically complexing with, or measuring the level of a biomarker selected from those of Table 1. In one embodiment, the composition includes 2, 3, 4, 5, 6, 7, 8, 9, 10, 11, 12, 13, 14, 15, 16, 17, 18, 19, 20, 21, 22, 23, 24, or 25 of said reagents. In another embodiment, a single set of reagents is provided which allows for detection or measurement of more than one biomarker selected from those of Table 1.

Reagents are provided herein which are capable of detecting, binding, specifically complexing with, or measuring the level of a subject biomarker. In one embodiment, the reagents are those which are capable of detecting, binding, specifically complexing with, or measuring the expression of a subject biomarker at the polypeptide or protein level. Such reagents are known in the art. In one embodiment, the reagent is an antibody or fragment thereof. In one embodiment, the antibody specifically binds to at least part of, i.e., a fragment or epitope of, the subject biomarker. Such fragments or epitopes include 8-15 amino acids, up to 25 aa, up to up to 75 aa, up to 100 aa, up to 150 aa, up to 200 aa, up to 300 aa, up to 400 aa, up to 500 aa, up to 600 aa, up to 700 aa, up to 750 aa. Such antibodies are known in the art, or may be developed.

Such reagents are useful in assays known to the person of skill in the art, such as an ELISA. Non-limiting examples of antibodies to the subject biomarkers are provided. However, it is noted that the sequence of each of the subject biomarkers are known. The person of skill in the art would readily be able to generate suitable reagents, e.g., antibodies, using known techniques. See, e.g., Greenfield, E. A., Chapter 7: Generating Monoclonal Antibodies in Antibodies: a Laboratory Manual, Second Ed., 2014. Other reagents for assessing the levels of non-protein biomarkers are known in the art, and described herein.

Examples of antibodies/reagents used to detect the subject biomarkers are as follows:

TABLE 2
Exemplary antibodies/reagents

| | Seq | Exemplary | |
|---|---|---|---|
| Subject Biomarkers | ID # | Antibody/reagent | Source |
| zonulin | 1 | MBS706368 | MyBiosource |
| β-glucan | | GT003 | Glucatell Kit, CapeCod |
| LPS binding protein | 2 | DY870-05 | R&D systems |
| regenerating islet- | 3 | ELH-REG3A-1 | Ray Biotech |
| derived protein 3 | | | |
| alpha (REG3α) | | | |
| sCD14 | 4 | DY383-05 | R&D systems |
| myeloperoxidase | 5 | BMS2038INST | Thermo Fischer |
| (MPO) | | | |
| soluble CD163 | 6 | DY1607-05 | R&D systems |
| IL-6 | 7 | K15067L-2 | Meso Scale |
| IL-1β | 8 | K15067L-2 | Meso Scale |
| CRP | 9 | DY1707 | R&D systems |
| d-dimer | 10 | EHDDIMER | Thermo Fischer |
| galectin-3 | 11 | DY2045 | R&D systems |
| galectin-9 | 12 | DY1154 | R&D systems |
| C3a | 13 | BMS2089 | Thermo Fischer |
| GDF-15 | 14 | DGD150 | R&D systems |
| IL-10 | 15 | K15067L-2 | Meso Scale |
| MCP-2 | 16 | K15067L-2 | Meso Scale |
| IL-15 | 17 | K15067L-2 | Meso Scale |
| MIP-1α | 18 | K15067L-2 | Meso Scale |
| IL-22 | 19 | K15067L-2 | Meso Scale |
| TNF-α | 20 | K15067L-2 | Meso Scale |

β-glucan detection in plasma may be performed using Limulus Amebocyte Lysate (LAL) assay (Glucatell Kit, CapeCod; catalog #GT003). The Glucatell® assay kit is specific for (1→3)-β-D-glucan. The assay is based upon a modification of the Limulus Amebocyte Lysate (LAL) pathway.

In one embodiment, an increase in the level of one of the subject biomarkers, as compared to a control, is indicative of an increased risk of moderate or severe COVID-19 disease.

In another embodiment, an increase in the level of one of the subject biomarkers, as compared to a control, is indicative of an increased risk of Long-COVID.

Provided herein are therapeutic targets for severe COVID-19, including zonulin. Zonulin is the only known physiological modulator of the intestinal tight junctions.⁷³ Microbial dysbiosis and translocation enhance zonulin release, which in turn induces tight junction permeability, leading to more microbial translocation. This microbial translocation triggers inflammation, which promotes further gut leakiness.^{40,41} Increased intestinal permeability and serum zonulin levels have been observed during many inflammatory diseases, including Crohn's disease and celiac disease.^{74,75} Preventing zonulin-mediated increase in intestinal permeability by a zonulin receptor antagonist AT1001 (larazotide acetate) decreased the severity and incidence of several inflammation-associated diseases in pre-clinical and clinical studies.⁷⁶⁻⁸² Thus, in one embodiment, the subject biomarker comprises zonulin.

A second therapeutic target revealed by this work is citrulline. Citrulline levels were reduced in both moderate and severe COVID-19, and the citrulline metabolism and biosynthesis pathways were among the top metabolic pathways disrupted in severe COVID-19. Citrulline is an intermediate in arginine metabolism,₈₉ and a marker of gut and enterocyte function._{25,90} Disrupted citrulline metabolism, as we observed during severe COVID-19, has been associated with microbial dysbiosis and dysregulated intestinal function.₉₁ In addition to its role as a biomarker, citrulline has an important role in preserving gut barrier function. In an intestinal obstruction mouse model, pretreatment with a citrulline-rich diet preserved gut barrier integrity.₉₂ Thus, in one embodiment, the subject biomarker comprises citrulline. In one embodiment, a decrease in the level of citrulline, as compared to a control, is indicative of an increased risk of moderate or severe COVID-19 disease.

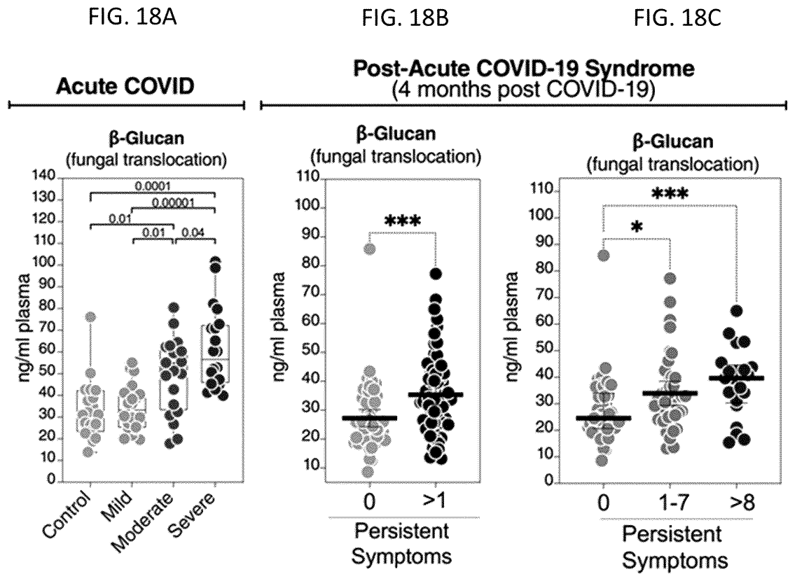

As described herein, higher levels of β-glucan were observed in the plasma of patients with Long COVID compared to non-Long-COVID (in a manner linked to the number of persistent symptoms; FIG. 18B-C), indicating elevated levels of fungal translocation during Long-COVID. β-glucan is a biomarker of lower gut integrity and higher microbial translocation during HIV infection, and its levels correlate with inflammation, immune suppression, and the development of HIV-associated comorbidities as shown in several reports. Thus, in one embodiment, the subject biomarker comprises β-glucan. In one embodiment, an increase in the level of β-glucan, as compared to a control, is indicative of an increased risk of Long-COVID.

In one embodiment, the subject biomarkers are selected from zonulin, LPS binding protein, β-glucan, and regenerating islet-derived protein 3 alpha (REG3α).

In another embodiment, the subject biomarkers are selected from sCD14, myeloperoxidase (MPO), soluble CD163, IL-6, IL-1β, CRP, d-dimer, galectin-3, galectin-9, C3a, and GDF-15.

In yet another embodiment, the subject biomarkers comprise zonulin, LBP, or β-glucan and one or more marker of inflammation.

In yet another embodiment, the subject biomarkers comprise one or more of CRP, IL-1, GDF-15, d-dimer, MPO, IP-10, IL-10, GAL-9, MCP-2, IL-15, MIP-1a, GAL-3, C3a, IL-1B, IL-22, TNF-a, IL-21, and fractalkine.

In yet another embodiment, the subject biomarkers comprise zonulin and one or more of CRP, IL-1, GDF-15, d-dimer, MPO, IP-10, IL-10, GAL-9, MCP-2, IL-15, MIP-1a, GAL-3, C3a, IL-1B, IL-22, TNF-a, IL-21, fractalkine, and IFN-γ.

In yet another embodiment, the subject biomarkers comprise LBP and one or more of CRP, IL-1, GDF-15, d-dimer, MPO, IP-10, IL-10, GAL-9, MCP-2, IL-15, MIP-1a, GAL-3, C3a, IL-1B, IL-22, TNF-α, and IFN-γ.

In yet another embodiment, the subject biomarkers comprise β-glucan and one or more of CRP, IL-1, GDF-15, d-dimer, MPO, IP-10, IL-10, GAL-9, MCP-2, MIP-1a, GAL-3, C3a, IL-1B, IL-22, and TNF-α.

In yet another embodiment, the subject biomarkers comprise zonulin, LBP and sCD14.

B. Compositions & Kits

The compositions, kits and methods described herein include reagents which are capable of detecting, binding, specifically complexing with, or measuring the level of the subject biomarker. Such reagents include those which are capable of detecting, or measuring the level of, said biomarker at the nucleic acid or protein level. In one embodiment, the reagents capable of detecting the biomarker(s) are proteins or polypeptides. In one embodiment, the proteins or polypeptides are antibodies or fragments thereof, e.g., such as those suitable for use in an ELISA. Further provided is a kit comprising the assay of the invention and optionally instructions for use.

In one embodiment, at least one reagent is labeled with a detectable label. Suitable labels include, without limitation, an enzyme, a fluorochrome, a luminescent or chemi-luminescent material, or a radioactive material. In another embodiment, at least one reagent is immobilized on a substrate.

In one embodiment, the assay is an enzyme-linked immunosorbent assay (ELISA), and the reagents are thus, appropriate for that format. In another embodiment, the suitable assay is selected from the group consisting of an immunohistochemical assay, a counter immuno-electrophoresis, a radioimmunoassay, radioimmunoprecipitation assay, a dot blot assay, an inhibition of competition assay, and a sandwich assay. In another embodiment, the assay is one that utilizes electrochemiluminescent detection. In another embodiment, the diagnostic reagent is labeled with a detectable label. In one embodiment, the label is an enzyme, a fluorochrome, a luminescent or chemi-luminescent material, or a radioactive material.

Any combination of the described reagents for the detection of the subject biomarkers can be assembled in a diagnostic kit for the purposes of diagnosing increased likelihood of moderate or severe COVID-19 disease. For example, one embodiment of a diagnostic kit includes reagents for 1, 2, 3, 4, 5, 6, 7, 8, 9, 10, 11, 12, 13, 14, 15, 16, 17, 18, 19, 20, 21, 22, 23, 24, or 25 of the subject biomarkers. In one embodiment, one or more of the reagents is associated or bound to a detectable label or bound to a substrate. For these reagents, the labels may be selected from among many known diagnostic labels, including those described above. Similarly, the substrates for immobilization may be any of the common substrates, glass, plastic, a microarray, a microfluidics card, a chip or a chamber.

Any combination of the described reagents for the detection of the subject biomarkers can be assembled in a diagnostic kit for the purposes of diagnosing increased likelihood of Long-COVID disease. For example, one embodiment of a diagnostic kit includes reagents for detection of β-glucan, optionally in combination with 1 or more other biomarkers. In one embodiment, one or more of the reagents is associated or bound to a detectable label or bound to a substrate. For these reagents, the labels may be selected from among many known diagnostic labels, including those described above. Similarly, the substrates for immobilization may be any of the common substrates, glass, plastic, a microarray, a microfluidics card, a chip or a chamber.

It is intended that any of the compositions described herein can be a kit containing multiple reagents or one or more individual reagents. For example, one embodiment of a composition includes a substrate upon which one or more of the reagents are immobilized. In another embodiment, the composition is a kit also contains optional detectable labels, immobilization substrates, optional substrates for enzymatic labels, as well as other laboratory items. In one embodiment, the kit contains a standard for use as a control.

C. Methods

In another aspect, methods of predicting increased risk of moderate or severe illness in a subject having, or suspected of having COVID-19, are provided. In one embodiment, the diagnostic method involves correlating the presence or amount of one or more subject biomarkers with a diagnosis of increased risk of moderate to severe illness. In one embodiment, the subject has tested positive for SARS-COV-2.

In one embodiment, the method includes detecting the level of one or more subject biomarker in a sample from a subject having, or suspected of having, an illness associated with a coronavirus infection and comparing the level of the one or more subject biomarkers of Table 1 to a control level. The method includes diagnosing the subject with an increased risk of moderate or severe respiratory illness when an increase in the level of one or more subject biomarkers is detected as compared to a control. In one embodiment, the method includes treating the subject for moderate or severe respiratory illness when an increased risk is detected, as described herein.

In another embodiment, the method includes diagnosing a subject with an increased risk of moderate or severe respiratory illness when an increase in the level of one or more subject biomarkers of Table 1 is detected as compared to a control. In one embodiment, the method includes treating the subject for moderate or severe respiratory illness when an increased risk is detected, as described herein. In one embodiment, the method includes use of one or more of the reagents described in Table 2.

In another embodiment, the method includes diagnosing a subject with an increased risk of developing Long-COVID, or having Long-COVID, when an increase in the level of one or more subject biomarkers of Table 1 is detected as compared to a control. In one embodiment, the method includes treating the subject for Long-COVID when an increased risk is detected, as described herein. In one embodiment, the method includes use of one or more of the reagents described in Table 2.

In one embodiment, the subject biomarkers are selected from zonulin, LPS binding protein, β-glucan, and regenerating islet-derived protein 3 alpha (REG3a).

In another embodiment, the subject biomarkers are selected from sCD14, myeloperoxidase (MPO), soluble CD163, IL-6, IL-1β, CRP, d-dimer, galectin-3, galectin-9, C3a, and GDF-15.

In yet another embodiment, the subject biomarkers comprise zonulin, LBP, or β-glucan and one or more marker of inflammation.

In yet another embodiment, the subject biomarkers comprise one or more of CRP, IL-1, GDF-15, d-dimer, MPO, IP-10, IL-10, GAL-9, MCP-2, IL-15, MIP-1a, GAL-3, C3a, IL-1B, IL-22, TNF-a, IL-21, and fractalkine.

In yet another embodiment, the subject biomarkers comprise zonulin and one or more of CRP, IL-1, GDF-15, d-dimer, MPO, IP-10, IL-10, GAL-9, MCP-2, IL-15, MIP-1a, GAL-3, C3a, IL-1B, IL-22, TNF-a, IL-21, fractalkine, and IFN-γ.

In yet another embodiment, the subject biomarkers comprise LBP and one or more of CRP, IL-1, GDF-15, d-dimer, MPO, IP-10, IL-10, GAL-9, MCP-2, IL-15, MIP-1a, GAL-3, C3a, IL-1B, IL-22, TNF-α, and IFN-γ.

In yet another embodiment, the subject biomarkers comprise β-glucan and one or more of CRP, IL-1, GDF-15, d-dimer, MPO, IP-10, IL-10, GAL-9, MCP-2, MIP-1a, GAL-3, C3a, IL-1B, IL-22, and TNF-α.

In yet another embodiment, the subject biomarkers comprise zonulin, LBP and sCD14.

In another embodiment, the method includes detecting the levels of metabolites in the plasma of a subject having, or suspected of having, illness associated with a coronavirus infection and comparing the levels of the metabolites to control levels. The method includes diagnosing the subject with a higher risk of moderate or severe respiratory illness when a significant change is detected in 10 or more metabolites in the subject's plasma as compared to a control. In one embodiment, the method includes treating the subject for moderate or severe respiratory illness when an increased risk is detected, as described herein. In one embodiment, the metabolites are those shown in Table 3.

TABLE 3
50 metabolites dysregulated by COVID-19

| Top 25 metabolites induced | SC | Top 25 metabolites reduced | SC |
|---|---|---|---|
| by severe COVID-19 | rho | by severe COVID-19 | rho |
| Glucose 6-phosphate | 0.81 | L-tryptophan | −0.74 |
| Adenosine monophosphate | 0.80 | 8-Hydroxyquinoline | −0.73 |
| N-acteylneuraminic acid | 0.80 | Skatole | −0.72 |
| Glucose 1-phosphate | 0.78 | 6-methylquinoline | −0.72 |
| Guanosine monophosphate | 0.75 | (2r)-2,3-dihydroxypropanoic | −0.69 |
| | | acid | |
| Xanthosine | 0.75 | Theophylline | −0.67 |
| N-acetyl-D-glucosamine | 0.75 | Catechol | −0.67 |
| Inosine | 0.74 | Citrulline | −0.67 |
| Cytidine monophosphate | 0.72 | 2,4-dihydroxybenzoic acid | −0.65 |
| Xanthine | 0.72 | L-histidine | −0.65 |
| L-cysteine-S-sulfate | 0.70 | Caffeine | −0.64 |
| Fructose 1,6-bisphosphate | 0.70 | D-(−)-Quinic acid | −0.64 |
| 3-Ureidopropionic acid | 0.69 | Indole-3-pyruvic acid | −0.63 |
| L-Lactic acid | 0.69 | L-Threonic acid | −0.62 |
| Maleic acid | 0.69 | 1,2-Dimethlyuric acid | −0.61 |
| D-Gluceraldehyde 3- | 0.69 | Methlycysteine | −0.59 |
| phosphate | | | |
| Inosinic acid | 0.68 | Trigonelline | −0.58 |
| Hypoxanthine | 0.67 | N-Acetylornithine | −0.54 |
| 11(Z),14(Z)-Eicosadienoic | 0.66 | Ornithine | −0.53 |
| Acid | | | |
| Gamma-Aminobutyric acid | 0.66 | 1-Nitrosopiperidine (NPIP) | −0.53 |
| L-Glutamic acid | 0.66 | 7-Methlyxanthine | −0.53 |
| 3-Phosphoglyceric acid | 0.65 | 2-Furoylglycine | −0.52 |
| 11(E)-Eicosenoic acid | 0.64 | Indole-3-acetic acid | −0.52 |
| D-2-Hydroxyglutaric acid | 0.63 | δ-Ribono-1,4-lactone | −0.51 |
| 4-hydroxybutyric acid | 0.63 | 2,3-Dihydroxybenzoic acid | −0.50 |
| (GHB) | | | |

In another embodiment, the method includes diagnosing a subject with an increased risk of moderate or severe illness when a decrease in the level of citrulline is detected as compared to a control. In one embodiment, the method includes treating the subject for moderate or severe respiratory illness when an increased risk is detected, as described herein.

In another embodiment, the method includes diagnosing a subject with an increased risk of moderate or severe illness when an increase in the level of succinic acid is detected as compared to a control. In one embodiment, the method includes treating the subject for moderate or severe respiratory illness when an increased risk is detected, as described herein.

In another embodiment, the method includes diagnosing a subject with an increased risk of moderate or severe illness when a significant increase is detected in the Kyn/Trp ratio in the subject's plasma as compared to a control. In one embodiment, the method includes treating the subject for moderate or severe respiratory illness when an increased risk is detected, as described herein.

In another embodiment of the methods described, the method further includes measuring the level of IL-6, where an increase in IL-6 is further indicative of increased risk of moderate or severe illness.

In another embodiment, a method for detecting an increased risk of death associated with COVID-19 disease in a subject is provided. The method includes detecting the level of one or more subject biomarkers of intestinal barrier integrity or inflammation in a sample from a subject having, or suspected of having, a respiratory illness associated with a coronavirus infection; comparing the level of the one or more subject biomarkers to a control level; and diagnosing the subject with an increased risk of death when an increase in the level of one or more subject biomarkers is detected as compared to a control. In one embodiment, the method includes treating the subject for moderate or severe respiratory illness when an increased risk is detected.

By significant increase, as used herein, means a statistically significant increase.

In certain embodiments, the methods provided include measuring one or more subject biomarkers in a biological sample. As described, altered levels of specific biomarkers are associated with an increased risk of moderate or severe illness in a patient. In certain embodiments, the method includes detection of zonulin in biological sample (e.g. serum), wherein an elevated level of zonulin is associated with an increased risk of moderate or severe illness in a subject. In certain embodiments, the elevated level is determined by comparison with a control (i.e., healthy) patient sample or with a sample obtained from a patient with mild illness. In certain embodiments, a patient with a serum concentration of zonulin greater than 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 125 ng/ml, 150 ng/ml, 175 ng/ml, 175 ng/ml, or 200 ng/ml is determined to be at increased risk of moderate or severe illness. In certain embodiments, an increased risk of moderate or severe illness is determined based on a serum concentration of zonulin of at least 100 ng/ml.

In further embodiments, an increased risk of moderate or severe illness in a patient is determined based on the detection of zonulin in a patient sample in combination with one or more subject biomarkers described herein. In certain embodiments, the method includes detecting zonulin in combination with LPS-binding protein (LBP) and/or soluble CD14 (sCD14). The individual subject biomarkers may be detected in the same biological sample or from multiple samples obtained from a subject or from samples aliquoted from a sample. In certain embodiments, a subject with elevated levels of serum zonulin in combination with a LBP serum concentration greater than 10,000 ng/ml, 15,000 ng/ml, 20,000 ng/ml, 25,000 ng/ml, 30,000 ng/ml, 35,000 ng/ml, or 40,000 ng/ml is determined to be at an increased risk of moderate or severe illness. In certain embodiments, the patient is determined to be at an increased risk of moderate or severe illness when a serum concentration of zonulin greater than 50 ng/ml is detected in combination with a serum concentration of LBP greater than 10,000 ng/ml. In certain embodiments, a subject with elevated levels of serum zonulin in combination with a sCD14 serum concentration greater than 2,000 ng/ml, 2,250 ng/ml, 2,500 ng/ml, 2,750 ng/ml, 3,000 ng/ml, 3,250 ng/ml, 3,500 ng/ml, 3,750 ng/ml, or 4,000 ng/ml is determined to be at an increased risk of moderate or severe illness. In certain embodiments, a subject is determined to at an increased risk of moderate or severe illness based on detection of elevated concentrations of zonulin, LBP, and sCD14 in serum obtained from the subject. In certain embodiments, a patient with an increased risk of moderate or severe illness is identified based on a serum concentration of zonulin greater than 50 ng/ml, a serum concentration of LPB greater than 20,000 ng/ml, and a serum sCD14 concentration greater than 2,000 ng/ml.

In certain embodiments, an increased risk of moderate or severe illness in a patient is determined based on the detection of a subject biomarker that is a kynurenine to tryptophan concentration ([kynurenine]/[tryptophan]) ratio in a serum sample. In certain embodiments, the elevated [kynurenine]/[tryptophan] ratio is determined by comparison with a control (i.e., healthy) patient sample or with a sample obtained from a patient with mild illness. In certain embodiments, a subject is determined to be at an increased risk of moderate or severe illness based on a serum [kynurenine]/[tryptophan] ratio greater than 0.10, greater than 0.15, greater than 0.20, greater than 0.25, or greater than 0.30. In a further embodiment, an increased risk of moderate or severe illness in a patient is determined based on an increased serum [kynurenine]/[tryptophan] ratio in combination and an increased serum zonulin concentration, relative to, e.g., levels detected in a healthy patient sample or a sample obtained from a patient with mild illness.

As described, in certain embodiments, altered levels of specific biomarkers are associated with an increased risk Long-COVID, or diagnosis of Long-COVID, in a patient. In certain embodiments, the method includes detection of β-glucan in biological sample (e.g. serum or plasma), wherein an elevated level of β-glucan is associated with an β-glucan in a subject. In certain embodiments, the elevated level is determined by comparison with a control (i.e., healthy) patient sample or with a sample obtained from a patient with mild illness. In certain embodiments, a patient with a serum concentration of β-glucan at least 25 ng/ml, 30 ng/ml, 31 ng/ml, 32 ng/ml, 33 ng/ml, 34 ng/ml, 35 ng/ml, 36 ng/ml, 37 ng/ml, 38 ng/ml, 39 ng/ml, 40 ng/ml, 45 ng/ml, 50 ng/ml, or 55 ng/ml is determined to be at increased risk of Long-COVID. In certain embodiments, an increased risk of Long-COVID is determined based on a serum concentration of β-glucan of at least 30 ng/ml.

In one embodiment, the amount of the one or more subject biomarkers is compared to one or more control levels, as described herein, to provide the diagnosis of increased risk. In one embodiment, the predetermined control may be tailored specifically for the sample being tested. For example, for the sample of an African American patient, a predetermined control comprising healthy African American subjects may be used. Alternatively, it may be advantageous to use, for example, the mean level of a predetermined control group comprising all subjects with a specific common characteristic, e.g., race or age. Alternatively, it may be advantageous to use, for example, the mean level of a predetermined control group comprising all healthy subjects with no COVID-19 disease. In another embodiment, the control is an artificial control, such as a standard provided with a kit.

In another embodiment, the subject is being treated for COVID-19 disease and the method enables a determination of the efficacy of the treatment. In one embodiment, the method involves detecting the level of one or more subject biomarkers, in a biological sample from a mammalian subject having COVID-19 disease over a selected time period. The level of the subject biomarkers is then compared with the level in one or more biological samples of the same subject assayed earlier in time, or before or during treatment. In one embodiment, the comparison can occur by direct comparison with one or more prior assessments of the same patient's status. In another embodiment, the reference may be a negative control comprising healthy subjects. The treatment for COVID-19 may be any described herein, or known in the art.

In one embodiment, the contacting step comprises forming a direct or indirect complex in the subject's biological sample between a diagnostic reagent for the subject biomarker and the subject biomarker in the sample. In yet another embodiment, the contacting step further comprises measuring a level of the complex in a suitable assay.

In one embodiment, the immunoassay is an enzyme-linked immunosorbent assay (ELISA). In another embodiment, the suitable assay is selected from the group consisting of an immunohistochemical assay, a counter immuno-electrophoresis, a radioimmunoassay, radioimmunoprecipitation assay, a dot blot assay, an inhibition of competition assay, and a sandwich assay.

In another embodiment, one or more of the diagnostic reagents is labeled with a detectable label. In one embodiment, the label is an enzyme, a fluorochrome, a luminescent or chemi-luminescent material, or a radioactive material. In another embodiment, the diagnostic reagent is an antibody or fragment thereof specific for one of the subject biomarkers.

Where applicable, the measurement of the subject biomarker(s) protein in the biological sample may employ any suitable ligand (reagent), e.g., antibody to detect the protein. Such antibodies may be presently extant in the art or presently used commercially, or may be developed by techniques now common in the field of immunology. As used herein, the term “antibody” refers to an intact immunoglobulin having two light and two heavy chains or any fragments thereof. Thus, a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain FAT construct, a Fab construct, a light chain variable or complementarity determining region (CDR) sequence, etc. As used herein, the term “antibody” may also refer, where appropriate, to a mixture of different antibodies or antibody fragments that bind to the subject biomarker. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds to one of the subject biomarkers as defined above may be employed in the methods of the present invention, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g., colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting protein-antibody complex in applicable assays. Still other labels include fluorescent compounds, radioactive compounds or elements. Preferably, an antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The antibodies or fragments useful in this invention are not limited by the particular detectable label or label system employed. Thus, selection and/or generation of suitable antibodies with optional labels for use in this invention is within the skill of the art, provided with this specification, the documents incorporated herein, and the conventional teachings of immunology.

In another aspect, a method of diagnosing and treating a subject for an increased risk of moderate to severe COVID-19 disease is provided. The method includes contacting a sample from the subject with a reagent capable of detecting, binding, specifically complexing with, or measuring the level of one of the subject biomarkers from Table 1. Such reagents are described herein. The subject is diagnosed increased risk of moderate or severe respiratory illness when an increase in the level of one or more subject biomarkers is detected as compared to a control. An effective amount of a therapeutic is administered to treat moderate or severe illness.

In one embodiment, therapies for treatment of COVID-19 disease are known in the art. Such therapies include, without limitation, oxygen therapy, antivirals such as remdesivir, dexamethasone (or other corticosteroid), and antibody therapy. Treatments for Long-COVID may include treatments for the individual symptoms being experienced by the patient. In addition, several types of medication have been identified as useful in the treatment of Long-COVID including, without limitation, glucocorticoid steroids, statins, and CCR5 inhibitors.

In one embodiment, therapy for treatment of COVID-19 disease includes a treatment to reduce gut permeability. In one embodiment, therapy includes a composition to reduce zonulin levels in the subject. In one embodiment, the therapy includes administration of a zonulin receptor antagonist or blocking the zonulin pathway. Certain zonulin receptor antagonists are known in the art and include, without limitation, larazotide acetate, INN-202, SPD-550, INN-217, and/or INN-289.

In another embodiment, therapy includes increasing the level of citrulline in the subject. In another embodiment, therapy includes inhibiting or reducing the level of one or more galectins in the subject. Suitable galectins include GAL-3 and GAL-9. In one embodiment, the therapy is a small molecule galectin inhibitor.

In another embodiment, therapy includes dietary changes to reduce gut permeability or increase citrulline levels.

In another aspect, a method of treating a subject having COVID-19 disease is provided. In one embodiment, the method includes administering a zonulin receptor antagonist. Zonulin receptor antagonists are known in the art, and include, without limitation, larazotide acetate, INN-202, SPD-550, INN-217, and/or INN-289. In another embodiment, the method includes increasing the level of citrulline level in the subject. In one embodiment, citrulline level is increased via dietary supplement or dietary source. Dietary sources of citrulline include watermelon, legumes, meat, and nuts.

In another embodiment, the method includes inhibiting or reducing the level of one or more galectins in the subject. In one embodiment, the galectin is GAL-3. In another embodiment, the galectin is GAL-9. In another embodiment, the method includes administering a small molecule galectin inhibitor. Such inhibitors are known in the art and include those discussed by Stegmeyr et al, Extracellular and intracellular small-molecule galectin-3 inhibitors, Sci Rep 9, 2186 (2019), which is incorporated herein by reference.

Unless defined otherwise in this specification, technical and scientific terms used herein have the same meaning as commonly understood by one of ordinary skill in the art to which this invention belongs and by reference to published texts.

It should be understood that while various embodiments in the specification are presented using “comprising” language, under various circumstances, a related embodiment is also described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

### V. Examples

The examples that follow do not limit the scope of the embodiments described herein. One skilled in the art will appreciate that modifications can be made in the following examples which are intended to be encompassed by the spirit and scope of the invention.

### Example 1: Example 1: Materials and Methods

### Study Cohort

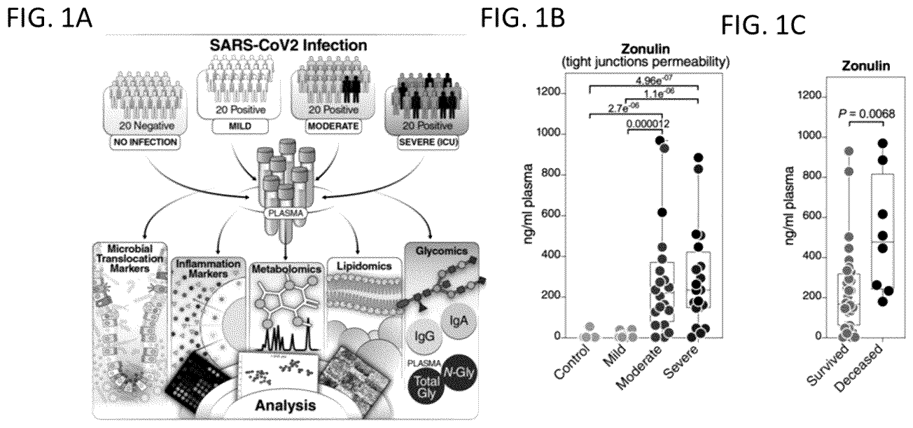

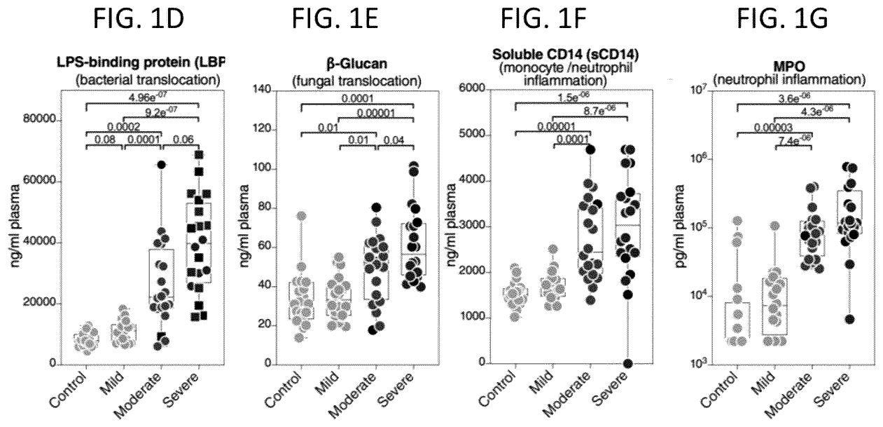

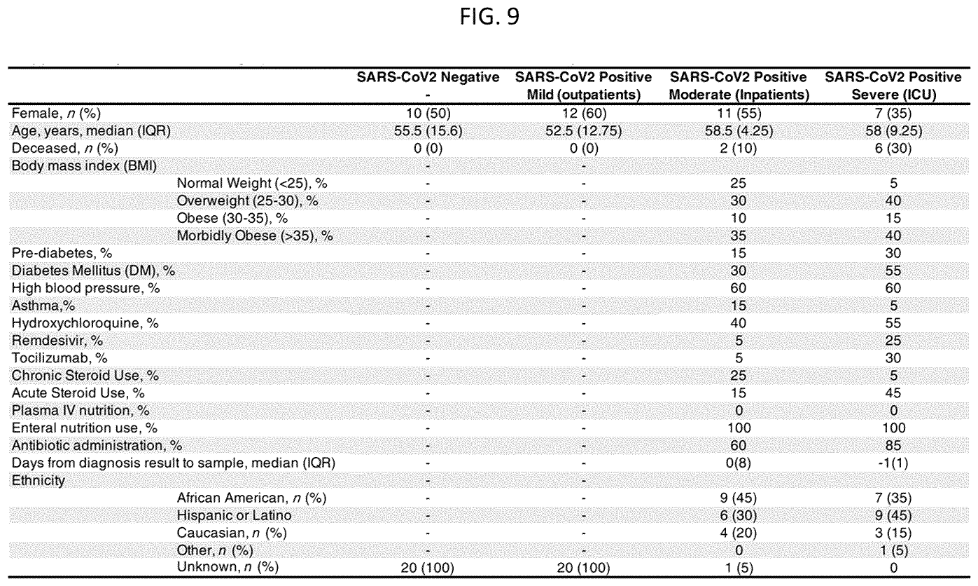

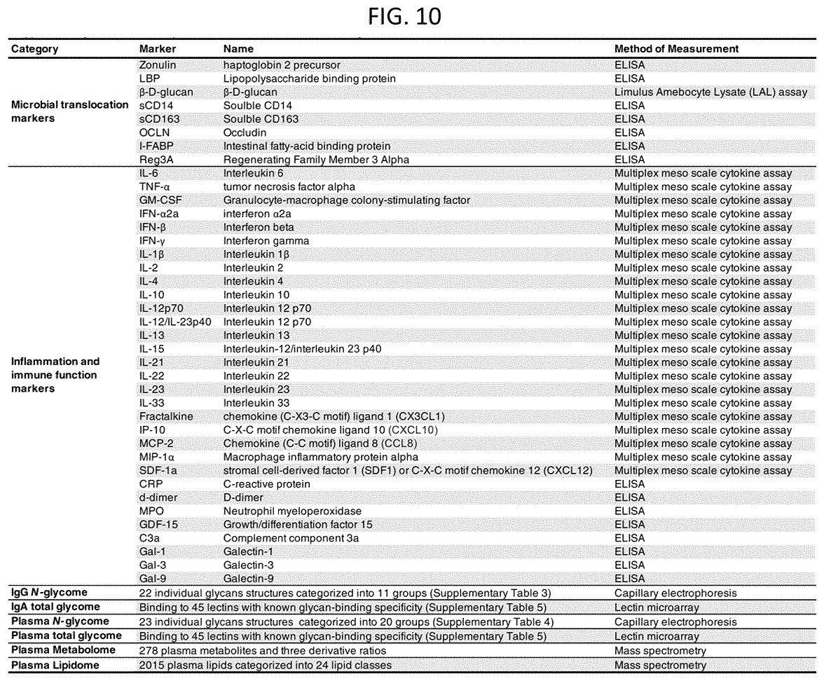

Analyses were performed using plasma samples from 60 individuals tested positive for SARS-CoV-2, and 20 SARS-CoV-2 negative controls collected at Rush University Medical Center (RUMC). The 60 SARS-CoV-2 positives were selected to represent three diseases states: 20 with mild symptoms (outpatients); 20 with moderate symptoms (inpatients hospitalized on regular wards); and 20 with severe symptoms (inpatients hospitalized in an intensive care unit (ICU)) (FIG. 1A). Individuals were selected to have a median age between 52.5 to 58.5 years. The study cohort was also chosen to have a 35 to 60% representation of female gender per disease status group (FIG. 9). Eight participants of the cohort (two from the moderate group and six from the severe group) died from COVID-19 (FIG. 9). All research protocols of the study were approved by the institutional review boards (IRB) at Rush University and The Wistar Institute. All human experimentation was conducted in accordance with the guidelines of the US Department of Health and Human Services and those of the authors' institutions.

### Study Validation Cohort

Key measurements [zonulin, LPS Binding Protein (LBP), and soluble CD14] were confirmed using plasma samples from an independent cohort of 57 individuals tested positive for SARS-CoV-2 and 18 SARS-CoV-2 negative controls collected at RUMC. The 57 SARS-CoV-2 positives were selected to represent three disease states: 20 with mild symptoms (outpatients); 18 with moderate symptoms (inpatients hospitalized on regular wards); and 19 with severe symptoms (inpatients hospitalized in an ICU).

### Measurement of Plasma Markers of Tight Junction Permeability and Microbial Translocation

Plasma levels of soluble CD14 (sCD14), soluble CD163 (sCD163), LPS Binding Protein (LBP), and FABP2/I-FABP were quantified using DuoSet ELISA kits (R&D Systems; catalog #DY383-05, #DY1607-05, #DY870-05, and #DY3078, respectively). The plasma level of zonulin was measured using an ELISA kit from MyBiosorce (catalog #MBS706368). Levels of occludin were measured by ELISA (Biomatik; catalog #EKC34871). β-glucan detection in plasma was performed using Limulus Amebocyte Lysate (LAL) assay (Glucatell Kit, CapeCod; catalog #GT003). Plasma levels of Reg3A were measured by ELISA (RayBiotech; catalog #ELH-REG3A-1).

### Measurement of Plasma Markers of Inflammation and Immune Activation

Plasma levels of GM-CSF, IFN-β, IFN-γ, IL-10, IL-13, IL-1β, IL-33, IL-4, IL-6, TNF-α, Fractalkine, IL-12p70, IL-2, IL-21, IL-22, IL-23, IP-10, MCP-2, MIP-1α, SDF-1a, IFN-α2a, IL-12/IL-23p40, and IL-15 were determined using customized MSD U-PLEX multiplex assay (Meso Scale Diagnostic catalog #K15067L-2). Plasma levels of C-Reactive Protein (CRP), Galectin-1, Galectin-3, and Galectin-9 were measured using DuoSet ELISA kits (R&D Systems; catalog #DY1707, #DY1152-#DY2045, and #DY1154, respectively). Levels of Growth Differentiation Factor-15 (GDF-15) were measured by ELISA using GDF-15 Quantikine ELISA Kit (R&D Systems; catalog #DGD150). Plasma levels of Myeloperoxidase (MPO), d-dimer, and C3a were measured by ELISA (Thermo Fischer; catalog #BMS2038INST, #EHDDIMER, #BMS2089, respectively).

### Untargeted Measurement of Plasma Metabolites

Metabolomics analysis was performed as described previously.¹⁰⁰ Briefly, polar metabolites were extracted from plasma samples with 80% methanol. A quality control (QC) sample was generated by pooling equal volumes of all samples and was injected periodically during the analysis sequence. LC-MS/MS was performed on a Thermo Scientific Q Exactive HF-X mass spectrometer with HESI II probe and Vanquish Horizon UHPLC system. Hydrophilic interaction liquid chromatography was performed at 0.2 ml/min on a ZIC-pHILIC column (2.1 mm×150 mm, EMD Millipore) at 45° C. Solvent A was 20 mM ammonium carbonate, 0.1% ammonium hydroxide, pH 9.2, and solvent B was acetonitrile. The gradient was 85% B for 2 min, 85% B to 20% B over 15 min, 20% B to 85% B over 0.1 min, and 85% B for 8.9 min. All samples were analyzed by full MS with polarity switching. The QC sample was also analyzed by data-dependent MS/MS with separate runs for positive and negative ion modes. Full MS scans were acquired at 120,000 resolution with a scan range of m/z. Data-dependent MS/MS scans were acquired for the top 10 highest intensity ions at 15,000 resolution with an isolation width of 1.0 m/z and stepped normalized collision energy of 20-40-60. Data analysis was performed using Compound Discoverer 3.1 (ThermoFisher Scientific). Metabolites were identified by accurate mass and retention time using an in-house database generated from pure standards or by MS2 spectra using the mzCloud spectral database (mzCloud.org) and selecting the best matches with scores of 50 or greater. Metabolite quantification used peak areas from full MS runs and were corrected based on the periodic QC runs.

### Untargeted Measurement of Plasma Lipids

Lipidomics analysis was performed as described previously.¹⁰¹ Briefly, plasma samples were spiked with EquiSplash mix (Avanti Polar Lipids). Lipids were extracted with 2:1:1 chloroform:methanol:0.8% sodium chloride. Samples were resuspended in 1:9 chloroform:methanol after drying the organic phase under nitrogen. LC-MS runs were performed on a Thermo Scientific Q Exactive HF-X mass spectrometer with HESI II probe and Vanquish Horizon UHPLC system. Reversed-phase liquid chromatography was performed at 0.35 ml/min on an Accucore C30 column (2.1 mm×150 mm, ThermoFisher Scientific) at 50° C. Solvent A was 50:50 acetonitrile:water, and solvent B was 88:10:2 isopropanol:acetonitrile:water solvents. Both solvents contained 5 mM ammonium formate and 0.1% formic acid. The gradient was 0% B for 3 min, 0% to 60% B over 7 min, 60% to 85% B over 10 min, 85% to 100% B over 10 min, 100% B for 5 min, 100% to 0% B over 0.01 min, and 0% B for 4.99 min. All samples were analyzed by data-dependent MS/MS with separate runs for positive and negative ion modes. Full MS scans were acquired at 120,000 resolution with a scan range of 300-2,000 m/z in positive mode and 250-2,000 m/z in negative mode. Data-dependent MS/MS scans were acquired for the top 20 ions at 15,000 resolution with an isolation width of 0.4 m/z. Stepped normalized collision energy of 20-30 was used for positive ion mode and 20-30-40 was used for negative ion mode. Data analysis was performed using LipidSearch 4.2 (ThermoFisher Scientific). Lipid species were identified from MS/MS spectra using an in-silico fragmentation database and were filtered by expected adducts and identification quality. Lipid species quantification used peak areas and was corrected based on EquiSplash deuterated lipids for represented classes.

### IgG Isolation

Bulk IgG was purified from plasma using Pierce Protein G Spin Plate (Thermo Fisher; catalog #45204). IgG was concentrated using Amicon® filters (Milipore catalogue #UFC805024) and purity was confirmed by SDS gel.

### IgA Isolation

Bulk IgA was purified from IgG depleted plasma using CaptureSelect IgA Affinity Matrix (Thermo Fisher; catalog #194288010). IgA purity was confirmed by SDS gel.

### N-Glycan Analysis Using Capillary Electrophoresis

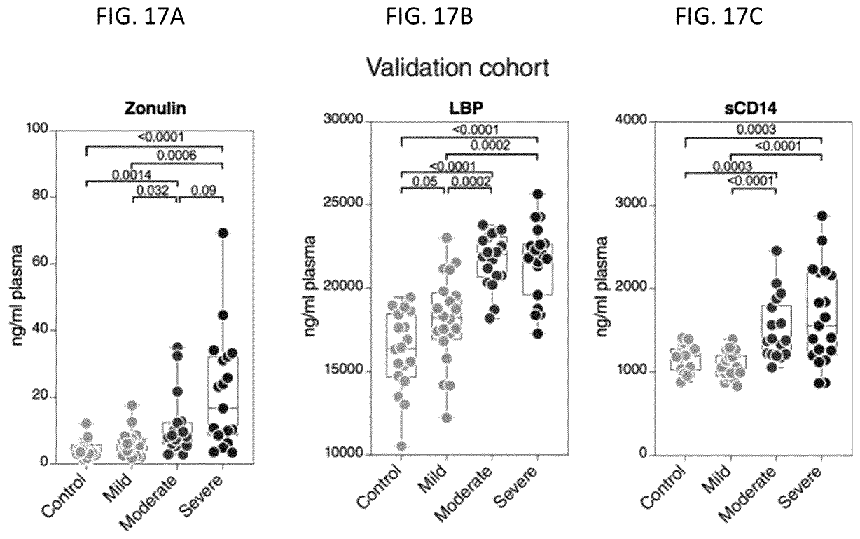

For both plasma and bulk IgG, N-glycans were released using peptide-N-glycosidase F (PNGase F) and labeled with 8-aminopyrene-1,3,6-trisulfonic acid (APTS) using the GlycanAssure APTS Kit (Thermo Fisher; catalog #A33952), following the manufacturer's protocol. Labeled N-glycans were analyzed using the 3500 Genetic Analyzer capillary electrophoresis system. Total plasma N-glycans were separated into 24 peaks (FIG. 17) and IgG N-glycans into 22 peaks (FIG. 18). The relative abundance of N-glycan structures was quantified by calculating the area under the curve of each glycan structure divided by the total glycans using the Applied Biosystems GlycanAssure Data Analysis Software Version 2.0.

### Glycan Analysis Using Lectin Array

To profile plasma total and IgA glycomes, we used the lectin microarray as it enables analysis of multiple glycan structures. The lectin microarray employs a panel of 45 immobilized lectins with known glycan-binding specificity (lectins and their glycan-binding specificity are detailed in FIG. 18). Plasma proteins or isolated IgA were labeled with Cy3 and hybridized to the lectin microarray. The resulting chips were scanned for fluorescence intensity on each lectin-coated spot using an evanescent-field fluorescence scanner GlycoStation Reader (GlycoTechnica Ltd.), and data were normalized using the global normalization method.

### Statistical Analysis

Kruskal-Wallis and Mann-Whitney U tests were used for unpaired comparisons. Spearman's rank correlations were used for bivariate correlation analyses. Severity correlation coefficient (SC rho) tested correlation versus patient groups with the severity groups quantified as follows: control=1, mild=2, moderate=3, severe=4. FDR for each type of comparison was calculated using the Benjamini-Hochberg approach within each data subset separately and FDR<5% was used as a significance threshold. Principal Component Analysis was performed on log 2-transformed z-scored data. Pathway enrichment analyses were done on features that passed significant SC rho at FDR<5%. Enrichments for the metabolites were tested using QIAGEN's Ingenuity® Pathway Analysis software (IPA®, QIAGEN Redwood City, www.qiagen.com/ingenuity) using the “Canonical Pathway” option. Enrichments for the lipids were done using LIPEA (https://lipea.biotec.tudresden.de/home) with default parameters. To explore biomarkers that could be distinguish clinical outcome (hospitalization vs. non-hospitalization), specific set of microbial translocation variables were identified among those with FDR<0.05. Variables for the multivariable logistic model were selected from the identified specific set of biomarkers using Lasso technique with the cross-validation (CV) selection option by separating data in 5-fold. Due to the exploratory nature of this study with moderate sample size, variable selection was determined using 100 independent rounds runs of CV Lasso with minimum tuning parameter lambda. The markers that were selected 80 or more times from 100 runs were used as final set of variables in our model. The ability of the final logistic model was assessed by AUC with 95% confidence interval. Statistical analyses were performed in R 4.0.2 and Prism 7.0 (GraphPad).

### Example 2: Severe COVID-19 is Fueled by Disrupted Gut Barrier Integrity

A disruption of the crosstalk between gut microbiota and the lung (gut-lung axis) has been implicated as a driver of severity during respiratory-related diseases. Lung injury causes systemic inflammation, which disrupts gut barrier integrity, increasing the permeability to gut microbes and their products. This exacerbates inflammation, resulting in positive feedback. To test the possibility that a disrupted gut contributes to Coronavirus disease 2019 (COVID-19) severity, we used a systems biological approach to analyze plasma from COVID-19 patients with varying disease severity and controls. Severe COVID-19 is associated with a dramatic increase in tight junction permeability and translocation of bacterial and fungal products from gut into blood. This disrupted gut barrier integrity correlates strongly with increased systemic inflammation and complement activation, lower gut metabolic function, and higher mortality. Our study highlights a previously unappreciated factor with significant clinical implications, disruption in gut barrier, as a force that contributes to COVID-19 severity.

### Characteristics of the Study Cohort and Study Overview

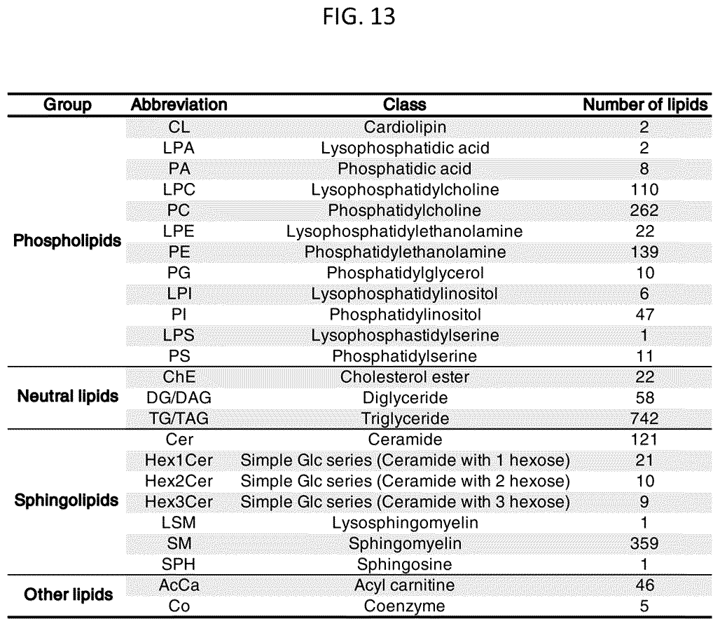

We collected plasma samples from 60 individuals testing positive for SARS-CoV-2 (by RT-PCR) and 20 SARS-CoV-2 negative controls. The 60 SARS-CoV-2 positive individuals were selected to represent three disease states: 20 with mild symptoms (outpatients); 20 with moderate symptoms (inpatients hospitalized on regular wards); and 20 with severe symptoms (inpatients hospitalized in an intensive care unit (ICU)) (FIG. 1A). Individuals were selected to have a median age between 52.5 to 58.5 years. There was no significant difference in age between groups. The study cohort was also chosen to have a 35 to 60% representation of female gender per disease status group (FIG. 9). Samples from hospitalized patients (moderate and severe groups) were collected at the time of diagnosis when the patient was admitted (FIG. 9). Eight individuals of the cohort (two from the moderate group and six from the severe group) died from COVID-19 (FIG. 9). The plasma samples from all individuals were used in a multi-omic, systems biology approach that measured: markers of tight junction permeability and microbial translocation using ELISA and Limulus Amebocyte Lysate assays; inflammation and immune activation/dysfunction markers using ELISA and multiplex cytokine arrays; untargeted metabolomic and lipidomic analyses using mass spectrometry (MS); and plasma glycomes (from total plasma glycoproteins, isolated immunoglobulin G (IgG), and isolated immunoglobulin A (IgA)) using capillary electrophoresis and lectin microarray (FIG. 1A and FIG. 13).

Severe COVID-19 is Associated with High Tight Junction Permeability and Microbial Translocation

We first asked whether severe COVID-19 is associated with differences in plasma markers tight junction permeability and microbial translocation. We measured the plasma levels of eight established drivers and markers of intestinal barrier integrity (FIG. 13). We found that severe COVID-19 is associated with high levels of zonulin (FIG. 1B). Zonulin (haptoglobin 2 precursor) is an established mediator of tight junction permeability in the digestive tract, where higher levels of zonulin drive increases in tight junction permeability.³⁹ Notably, hospitalized individuals with higher plasma levels of zonulin were more likely to die compared to hospitalized individuals with lower levels of zonulin (FIG. 1C).

These higher levels of zonulin could enable the translocation of microbes and their products from the gut into the blood, including parts of the cell wall of bacteria and fungus.^{40,41} To test this supposition, we measured plasma levels of common bacterial and fungal markers. Exposure to bacterial endotoxin can be determined by measuring plasma lipopolysaccharide (LPS) binding protein (LBP). LBP is an acute-phase protein that binds to LPS to induce immune responses.⁴² Indeed, we observed high levels of LBP in individuals with severe COVID-19 compared to individuals with mild COVID-19 or controls (FIG. 1D). We also found higher levels of β-glucan, a polysaccharide cell wall component of most fungal species and a marker of fungal translocation, 43 in individuals with severe COVID-19 compared to those with mild COVID-19 or controls (FIG. 1E). In addition, there were significantly higher levels (FDR=0.025) of the tight junction protein occludin in the severe group compared to controls (data not shown). There also was a strong trend (FDR=0.051) toward higher levels of Regenerating Family Member 3-Alpha (REG3a), a marker of intestinal stress,⁴⁴ comparing the severe and mild groups (data not shown). We did not observe high levels of intestinal fatty-acid binding protein (I-FABP), a marker of enterocyte apoptosis, suggesting that the high levels of tight junction permeability and microbial translocation are not associated with enterocyte death.

These high levels of tight junction permeability and microbial (both bacterial and fungal) translocation are expected to lead to microbial-mediated myeloid inflammation. Indeed, levels of soluble CD14 (sCD14; monocyte inflammation marker) (FIG. 1F) and myeloperoxidase (MPO; neutrophil inflammation marker) (FIG. 1G) were significantly higher during severe COVID-19, compared to mild and control groups. Levels of soluble CD163 (sCD163) were also higher significantly (FDR=0.04) in the severe group compared to controls (data not shown). These data indicate that COVID-19 severity and mortality are associated with plasma markers of higher tight junction permeability and higher translocation of bacterial and fungal products to the blood.

### Microbial Translocation is Linked to Systemic Inflammation

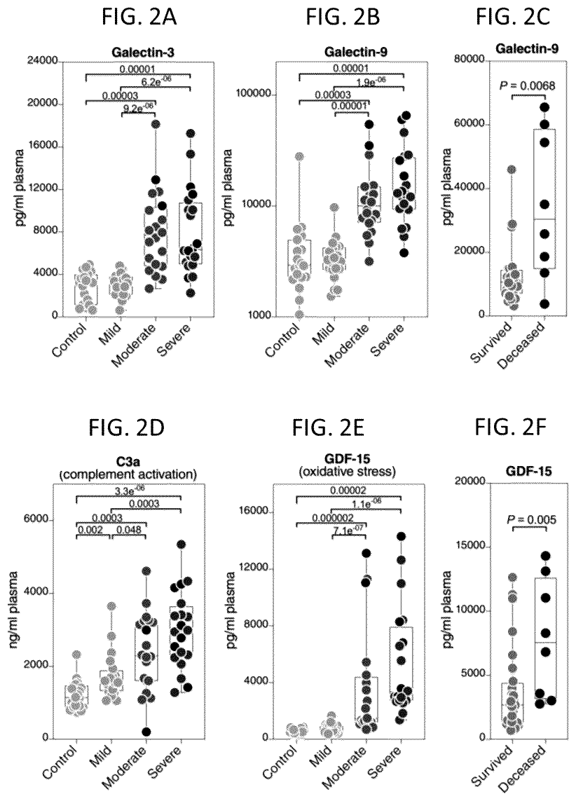

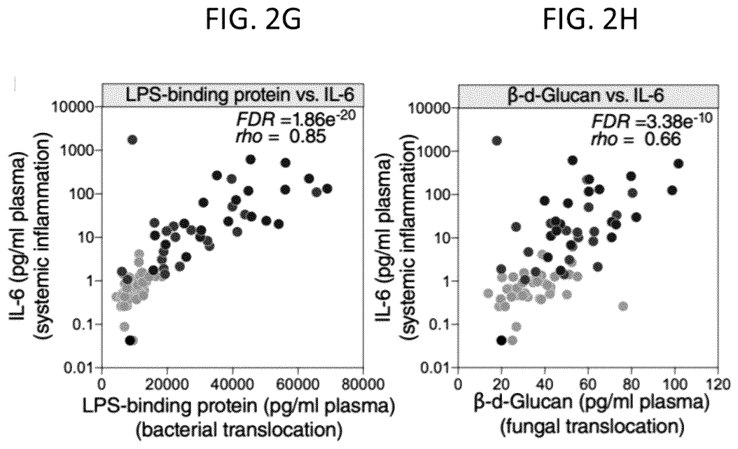

Higher levels of microbial translocation should lead to higher systemic inflammation. We measured the levels of 31 markers of systemic inflammation (FIG. 13) including: 23 cytokines and chemokines (such as IL-6, IL-1β, MCP-1, IP-10, and TNF-α), markers of inflammation and thrombogenesis (such as C-reactive protein (CRP) and D-dimer), a marker of complement activation (C3a), a marker of oxidative stress (GDF-15), and three immunomodulatory galectins (galectin-1, -3, and -9). As anticipated, many of these markers were higher in patients with severe COVID-19 compared to patients with mild COVID-19 or controls. In particular, we observed higher levels of several cytokines (such as IL-6 and IL-1β) and inflammatory markers (such as CRP and d-dimer). In addition to the expected changes, we also observed significant inductions in the immunomodulatory lectins galectin-3 (FIG. 2A) and galectin-9 (FIG. 2B). Levels of Gal-9 were higher in the plasma of hospitalized patients who eventually died compared to survivors (FIG. 2C). Last, notable dysregulations were observed in levels of C3a (FIG. 1D; indicative of complement activation) and GDF-15 (FIG. 1E; indicative of oxidative stress), with the levels of GDF-15 higher in deceased hospitalized patients compared to survivors (FIG. 1F).

Next, we examined the correlations between the markers of intestinal barrier integrity (zonulin) or microbial translocation (LBP and β-glucan) and the 31 markers of systemic inflammation and immune activation. Higher levels of zonulin, LBP, or β-glucan were strongly positively correlated with higher levels of many of the markers of systemic inflammation and immune activation, including IL-6 (FIG. 2G-FIG. 2H). These data support our hypothesis that disruption of intestinal barrier integrity, and microbial translocation during severe COVID-19 is linked to higher systemic inflammation and immune activation during severe COVID-19.

Severe COVID-19 is Associated with a Plasma Metabolomic Profile that Reflects Disrupted Gut Function

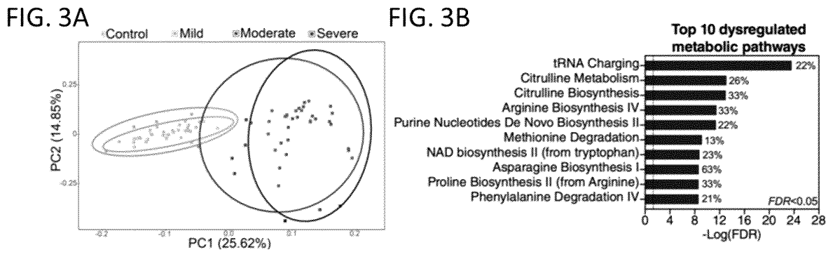

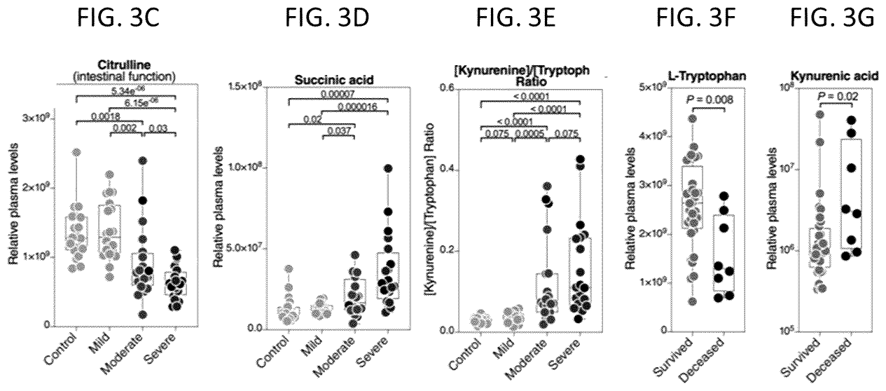

A second set of factors that reflect the functional state of the gut and its microbiota are the plasma metabolites. Importantly, many of these are not solely biomarkers of gut function/dysfunction, but also are biologically active molecules which can directly impact immunological and inflammatory responses. We performed untargeted metabolomic analysis (using LC-MS/MS. Within the 80 plasma samples, we identified a total of polar 278 metabolites. We observed a significant metabolomic shift during severe COVID-19 (FIG. 3A, a list of the top 50 dysregulated metabolites is in Table 3). Statistical significance was determined using the Kruskal-Wallis test. FDR was calculated using Benjamini-Hochberg method. SC rho=coefficient of correlation with COVID-19 severity.

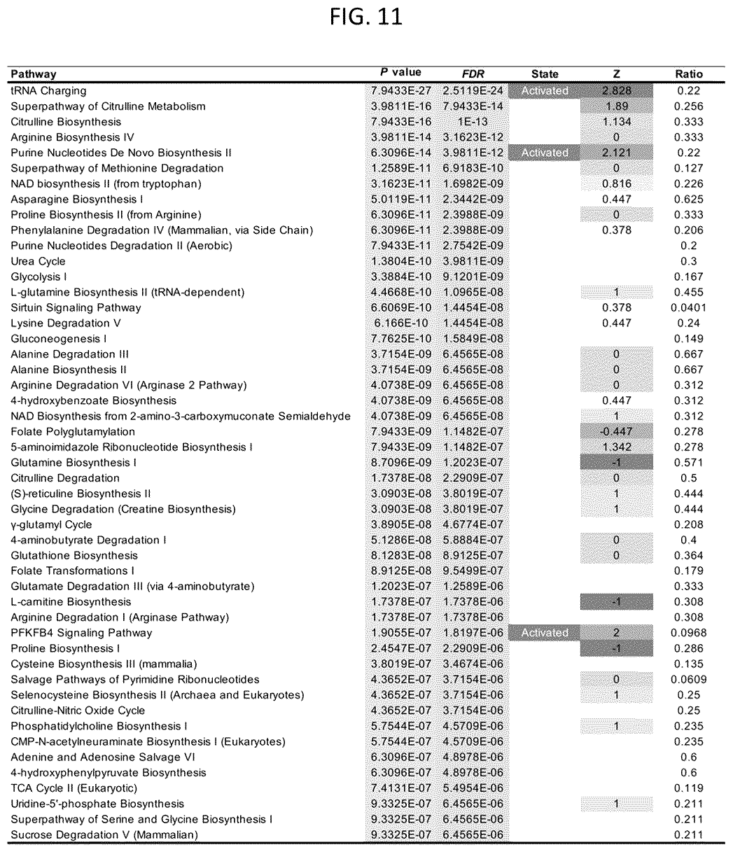

Indeed, in principal component analysis of the full metabolomic dataset, the first component was able to completely distinguish controls (and mild patients) from those with severe disease. Pathway analysis of the COVID-19-dysregulated metabolites showed disruption in tRNA charging, citrulline metabolism, and several other amino acid (AA) metabolic pathways (FIG. 3B, the top 10 dysregulated metabolic pathways are shown; FIG. 11 shows the top 50 dysregulated metabolic pathways with FDR<0.05). Importantly, changes in AA metabolism, including citrulline, arginine, methionine, and tryptophan (see FIG. 3B), are not only markers for gut dysfunction but also can influence the AA-metabolizing bacterial communities and disrupt the gut-microbiome immune axis.^{45,46} AA are absorbed and metabolized by enterocytes and gut microbiota. Consumption of AA by the gut microbiome is important for bacterial growth and is involved in the production of key microbiome-related metabolites.⁴⁶ These metabolites can influence epithelial physiology and be sensed by immune cells to modulate the mucosal immune system.^{47,48}

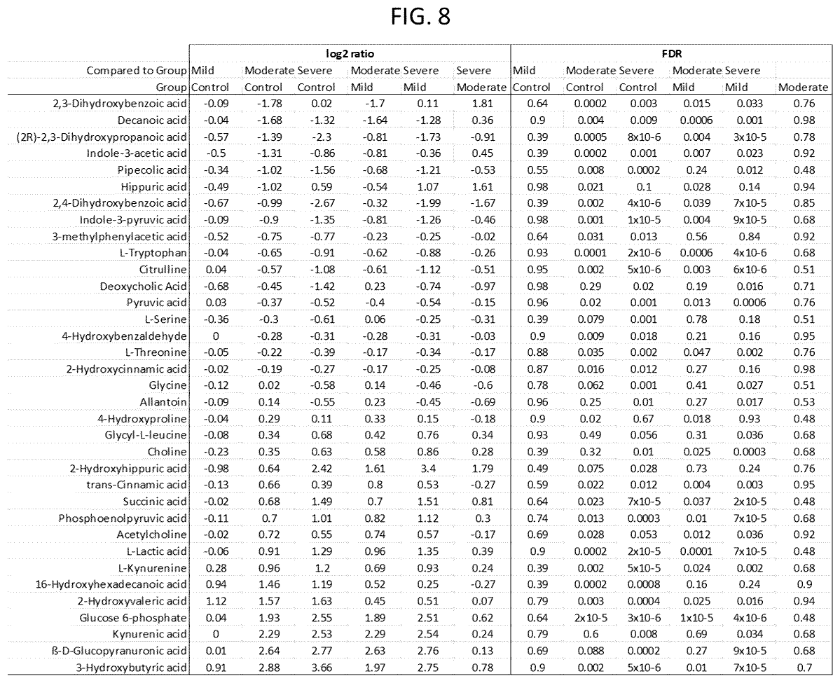

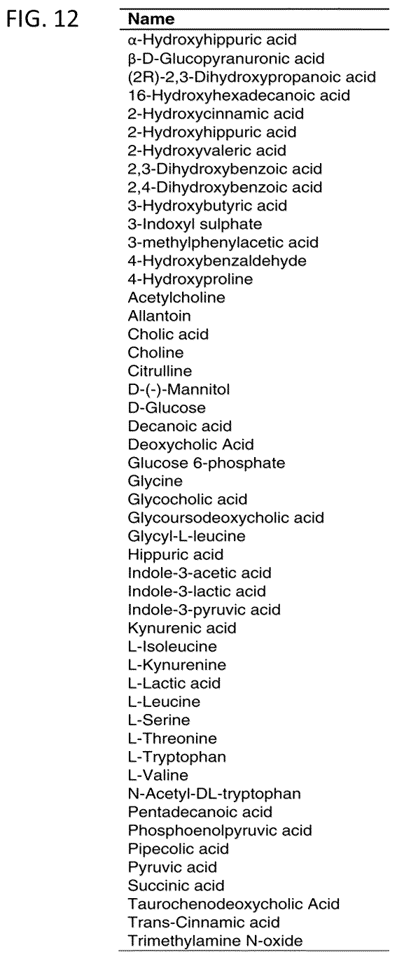

Next, we focused on 50 of the metabolites (out of the total of 278) that are known to be associated with the function of the gut and its microbiota (FIG. 12 lists the 50 metabolites and their references). Levels of most of these gut-associated plasma metabolites (35 out of 50) were dysregulated during severe COVID-19 compared to mild disease or controls (FIG. 8). Within this metabolic signature of COVID-19-associated gut dysfunction is citrulline, which is also identified as a top metabolic pathway dysregulated by severe COVID-19. Citrulline is an amino acid produced only by enterocytes and an established marker of gut and enterocyte function.²⁵ Its levels are significantly decreased during severe COVID-19 (FIG. 3C). Also, within this metabolic signature is succinic acid, a well-established marker of gut microbial dysbiosis, whose levels are higher during severe COVID-19 (FIG. 3D).

Notable differences were also observed in several metabolites involved in the catabolism of the AA tryptophan (FIG. 3B). Higher levels of tryptophan catabolism, indicated by high levels of kynurenine and low levels of tryptophan (i.e. the [Kyn/Trp] ratio), is an established marker of gut microbial dysbiosis.^{49,50} Indeed, we observed a higher [Kyn/Trp] ratio in individuals with severe COVID-19 than in those with mild disease or controls (FIG. 3E). Furthermore, lower levels of tryptophan and higher levels of kynurenic acid were associated with mortality among hospitalized COVID-19 patients (FIG. 3F-FIG. 3G). Together, these data indicate that a metabolic signature associated with severe COVID-19 is compatible with disrupted gut functions and dysregulated gut-microbiome axis. However, it is important to note that many of these metabolic pathways are multi-faceted and can also reflect dysregulations in multiple-organ systems.

Plasma Metabolomic Markers of COVID-19-Associated Gut Dysfunction Associate with Higher Inflammation and Immune Dysfunction

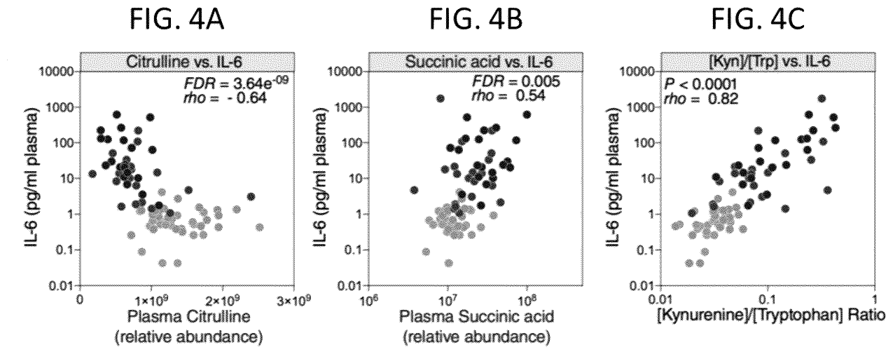

As noted above, many plasma metabolites are bioactive molecules that can directly impact immunological and inflammatory responses. Therefore, we sought to identify links between the 35 dysregulated gut-associated plasma metabolites (8) and the dysregulated markers of microbial translocation, inflammation, and immune activation. We observed strong links between levels of the dysregulated gut-associated metabolites and levels of markers of microbial translocation (data not shown) as well as levels of inflammation and immune activation (data not shown). Notable correlations were observed between lower levels of citrulline and higher IL-6 (FIG. 4A), higher levels of succinic acid and higher IL-6 (FIG. 4B), and higher [Kyn/Trp] ratio and higher IL-6 (FIG. 4C). These data highlight the potential links between disrupted metabolic activities, especially those related to the gut and its microbiota, and systemic inflammation and immune dysfunction during COVID-19.

Severe COVID-19 is Associated with Disrupted Lipid Metabolism

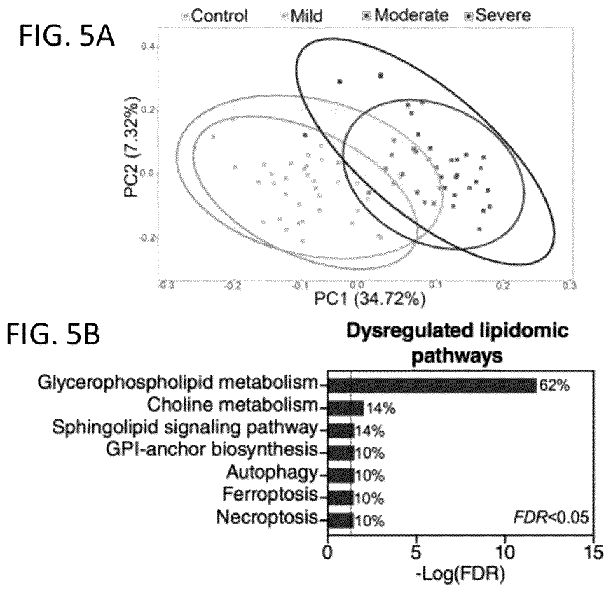

Intermediary metabolites and sulfur-containing amino acids (e.g. methionine, a regulated COVID-19 pathway, FIG. 3B) are potent modulators of lipid metabolism. Therefore, we performed lipidomic analysis on the plasma samples of the same cohort. We identified a total of 2015 lipids using untargeted MS. Similar to the plasma metabolome, the plasma lipidome shifted significantly during severe COVID-19 (FIG. 5A). These 2015 lipids were divided into 24 lipids classes (FIG. 13); out of these 24 classes, 16 were significantly (FDR<0.05) different in the moderate and severe COVID-19 groups (11 were lower whereas five were higher compared to the mild or control groups) (Data not shown). Pathway analysis of this severe-COVID-19-associated lipidomic signature showed that glycerophospholipid and choline metabolism were the most significantly dysregulated pathways (FIG. 5B). The gut microbiota is heavily involved in these two interconnected pathways.⁵¹ Gut microbial dysbiosis can alter the digestion and absorption of glycerophospholipids, leading to several diseases.⁵¹⁻⁵⁴ These data provide yet another layer of evidence that severe COVID-19 is associated with systemic dysregulations that are linked to disrupted gut function.

It is also known that COVID-19 severity is linked to pre-existing cardiometabolic-associated diseases²⁷. Furthermore, COVID-19 itself can cause liver dysfunction-. Indeed, many of the individuals in our main cohort with moderate and severe COVID-19 had diabetes and/or high blood pressure. We sought to examine whether these conditions contribute to our main findings. We examined the differences in the levels of zonulin, LBP, β-glucan, sCD14, and IL-6 between hospitalized patients (moderate and severe groups) who had diabetes or not, or patients who had high blood pressure or not. We did not observe any significant difference in the levels of these selected markers between these groups. However, the contribution of pre-existing metabolic conditions and post-infection intestinal and liver complications to the observed disrupted plasma profiles warrant further investigation.

Severe COVID-19 is Associated with Altered Plasma Glycomes that are Linked to Inflammation and Complement Activation.

Finally, we examined plasma glycomes. It has been reported that translocation of glycan-degrading enzymes released by several members of the gut microbiome can alter circulating glycomes. 32 Within the plasma glycome, glycans on circulating glycoproteins and antibodies (IgGs and IgAs) play essential roles in regulating several immunological responses, including complement activation. 31 For example, galactosylated glycans link Dectin-1 to Fcγ receptor IIB (FcγRIIB) on the surface of myeloid cells to prevent inflammation mediated by complement activation.³¹ A loss of galactose decreases the opportunity to activate this anti-inflammatory checkpoint, thus promoting inflammation and complement activation, including during IBD.^{32,34-37} Indeed, IgG glycomic alterations associate with IBD disease progression and IBD patients have lower IgG galactosylation compared to healthy controls.³³

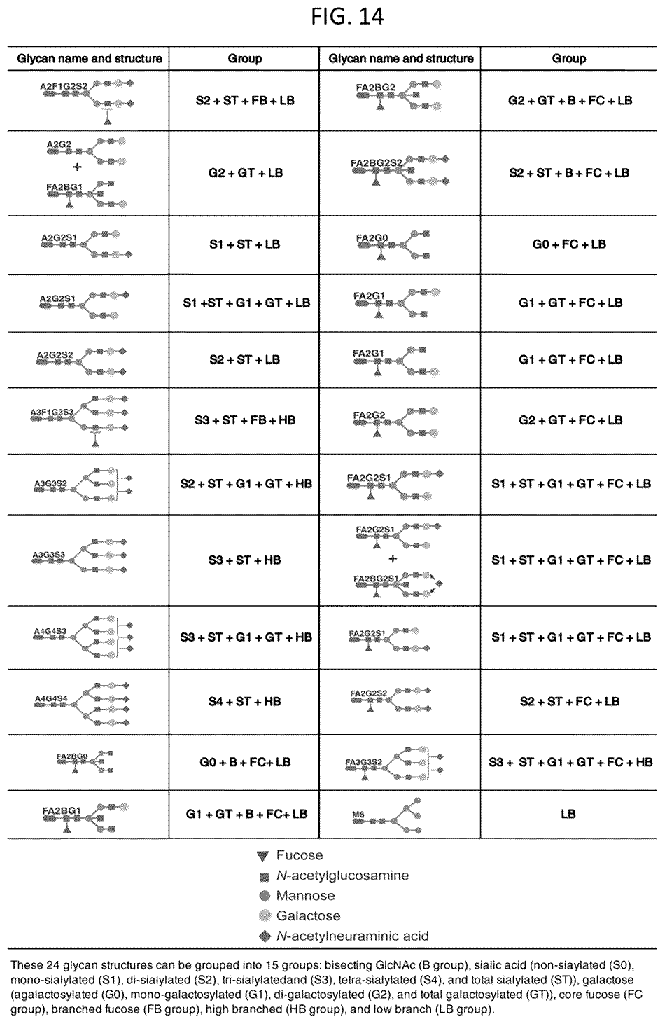

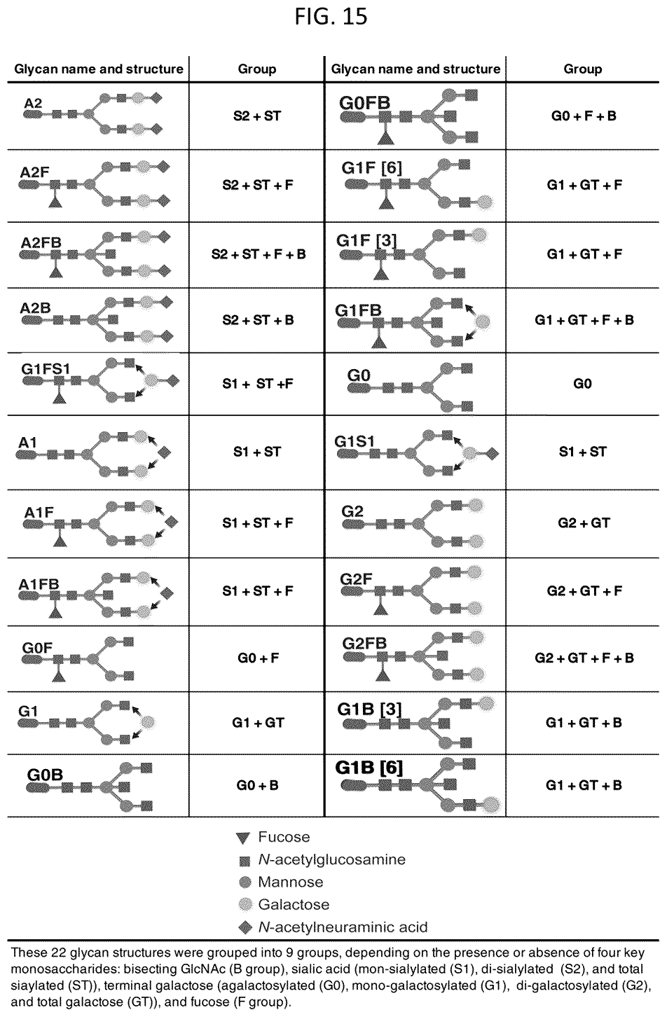

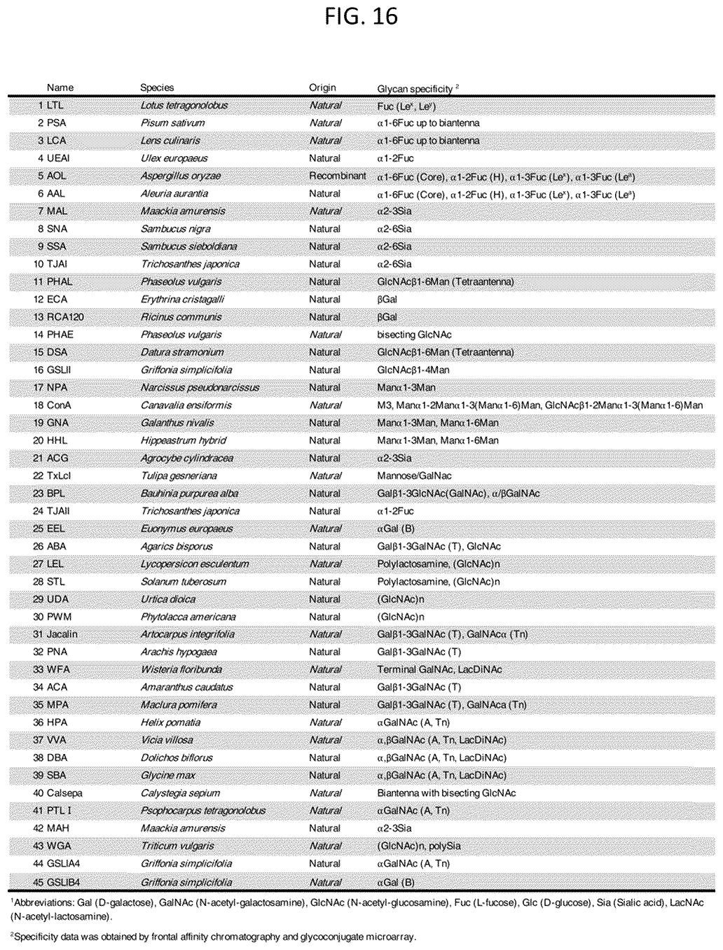

We applied several glycomic technologies to analyze the plasma glycome (total plasma, isolated IgG, and isolated IgA). First, we used capillary electrophoresis to identify the N-linked glycans of total plasma glycoproteins and isolated plasma IgG (this identified 24 and 22 glycan structures, respectively; their names and structures are in FIG. 14 and FIG. 15). We also used a 45-plex lectin microarray to identify other glycans on total plasma glycoproteins and isolated IgA. The lectin microarray enables sensitive analysis of multiple glycan structures by employing a panel of 45 immobilized lectins (glycan-binding proteins) with known glycan-binding specificity (FIG. 16 lists the 45 lectins and their glycan-binding specificities).⁵⁵

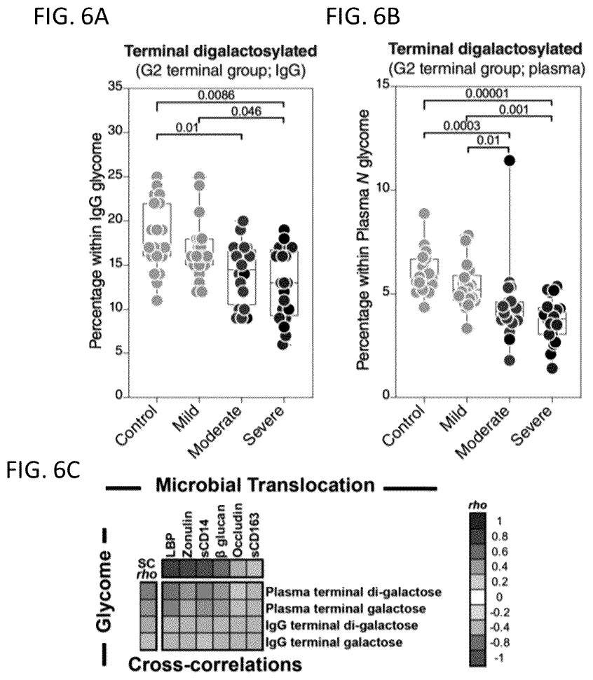

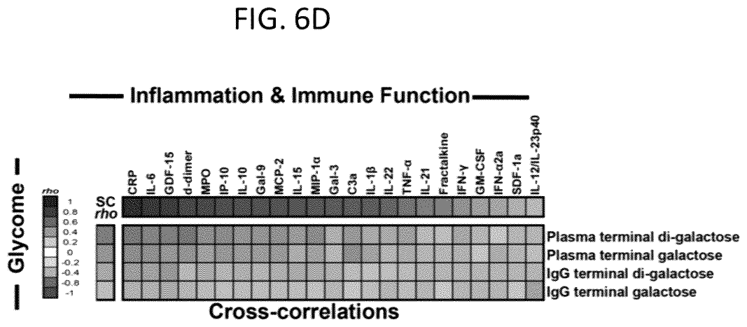

We first observed significant (FDR<0.05) glycomic differences during severe COVID-19 in levels of IgA glycans, plasma N-glycans, plasma total glycans, and IgG glycans. These changes are exemplified by an apparent loss of the anti-complement activation galactosylated glycans from IgG and total plasma glycoproteins (FIG. 6A-FIG. 6B, respectively). When we examined the correlations between the plasma glycome and markers of tight junction permeability/microbial translocation or inflammation/immune activation, as expected, we observed significant negative correlations (FDR<0.05) between levels of terminal galactose on IgG or plasma glycoproteins and markers of permeability/translocation (FIG. 6C) or markers of inflammation (FIG. 6D). These data highlight the potential links between the disrupted plasma glycome and systemic inflammation during COVID-19.

Multivariable Logistic Models, Using Cross-Validation Lasso Technique, Selected Gut-Associated Variables Whose Combination Associates with the Risk of Hospitalization During COVID-19

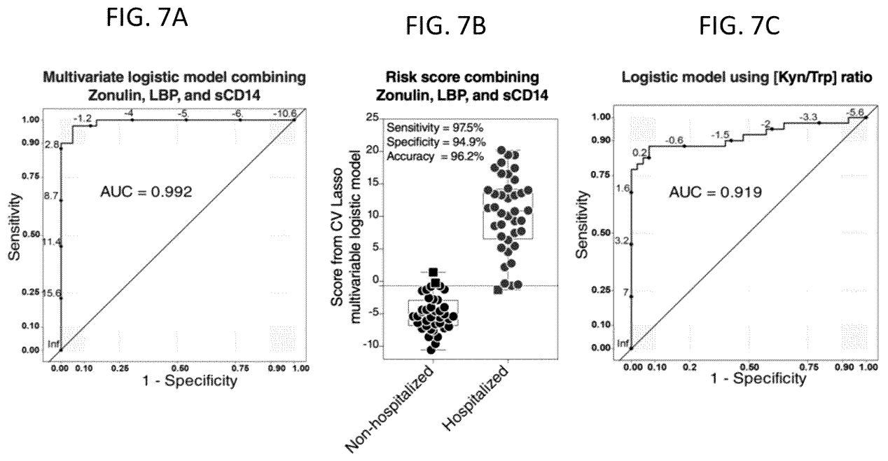

Our data thus far support the hypothesis that gut dysfunction fuels COVID-19 severity. We sought to examine whether markers of tight junction permeability and microbial translocation (FIG. 13) can distinguish between hospitalized COVID-19 patients (moderate and severe groups combined) and non-hospitalized individuals (mild and controls combined). We applied the machine learning algorithm Lasso (least absolute shrinkage and selection operator) regularization to select markers with the highest ability to distinguish between the two groups. The analysis employed samples with complete data sets (n=79; one sample did not have complete data). Lasso selected zonulin, LBP, and sCD14 as the three markers to be included in a multivariable logistic regression model that distinguishes hospitalized from non-hospitalized individuals with area under the ROC curve (AUC) of 99.23% (FIG. 7A; 95% confidence interval: 98.1%-100%). This value was higher than the AUC values obtained from logistic models using each variable individually (Table 4).

TABLE 4

| Variables | AUC | SE | 95% | CI | Pvalue |
|---|---|---|---|---|---|
| Variables | 0.992 | 0.01 | 0.981 | 1.000 | reference |
| identified by | | | | | |
| Lasso | | | | | |
| multivariable | | | | | |
| logistic model | | | | | |
| Zonulin | 0.951 | 0.03 | 0.901 | 1.000 | 0.0656 |
| LBP | 0.944 | 0.03 | 0.885 | 1.000 | 0.0987 |
| βglucan | 0.841 | 0.05 | 0.748 | 0.933 | 0.0008 |
| sCD14 | 0.930 | 0.03 | 0.871 | 0.988 | 0.0323 |
| Occludin | 0.663 | 0.06 | 0.542 | 0.783 | <0.0001 |
| Reg3A | 0.635 | 0.06 | 0.510 | 0.759 | <0.0001 |
| sCD163 | 0.627 | 0.06 | 0.503 | 0.751 | <0.0001 |
| IFABP | 0.538 | 0.07 | 0.408 | 0.668 | <0.0001 |

Bold variables are variables selected by Lasso to be included in the multivariable logistic model; AUC, Area under the ROC Curve; SE, Standard error. P-value (single predictor models vs. Lasso selected multivariable model)

Next, we used the multivariable logistic model to estimate a risk score of hospitalization for each individual. We then examined the ability of these risk scores to classify hospitalized from non-hospitalized individuals. As shown in FIG. 7B, the model correctly classified 97.5% of hospitalized (sensitivity) and 94.9% of non-hospitalized (specificity) individuals, with an overall accuracy of 96.2%. Furthermore, we examined the ability of the L-kynurenine/L-tryptophan [Kyn/Trp] ratio, an established marker of gut microbial dysbiosis described above, to distinguish hospitalized from non-hospitalized individuals. Logistic model showed that [Kyn/Trp] ratio alone can distinguish hospitalized from non-hospitalized with an AUC value of 91.9% (FIG. 7C; 95% confidence interval: >85%-98.7%). This analysis further highlights the plausible link between severe COVID-19 and disrupted gut function, orchestrated by an increase in tight junction permeability, microbial translocation, possible microbial dysbiosis, and dysregulated digestion and metabolism.

Zonulin, LBP, and sCD14 Plasma Levels are Higher During Severe COVID-19 in an Independent Validation Cohort

Finally, we sought to confirm some of our key findings in an independent cohort of 57 individuals tested positive for SARS-CoV-2 and 18 SARS-CoV-2 negative controls. The 57 SARS-CoV-2 positives were selected to represent three disease states: 20 with mild symptoms (outpatients); 18 with moderate symptoms (inpatients hospitalized on regular wards); and 19 with severe symptoms (inpatients hospitalized in an ICU). We focused on three measurements, zonulin, LBP, and sCD14, as these three measurements together were able to distinguish hospitalized from non-hospitalized individuals in the main cohort (FIGS. 7A-C). We observed higher levels of zonulin, LBP, and sCD14 during severe COVID-19 in this validation cohort (FIG. 17A-C). Furthermore, we validated our multivariable logistic model in FIG. 7A-C using data from this validation cohort. A combination of zonulin, LBP, and sCD14 was able to distinguish hospitalized from non-hospitalized individuals in the validation cohort with AUC of 88.6% (95% confidence interval: 80.3%-96.8%; Table 5). This analysis further highlights the plausible link between severe COVID-19 and disrupted gut function.

TABLE 5
AUC values of the logistic regression model built with data from the
main cohort and validated with data from the validation cohort.

| Cohort | n | AUC | SE | 95% Confidence interval |
|---|---|---|---|---|
| Main (training) | 79 | 0.9923 | 0.0057 | 0.981-1 |
| Validation | 75 | 0.886 | 0.042 | 0.803-0.968 |

We used a systems biology approach to provide multiple layers of evidence that severe COVID-19 is associated with markers of disrupted intestinal barrier integrity, microbial translocation, and intestinal dysfunction. These data highlight disruption in gut barrier integrity as a potential force that likely fuels COVID-19 severity. Our data are compatible with previous reports showed that severe COVID-19 is associated with bacterial translocation to the blood and increased levels of microbial-associated immune activation markers. Our results do not imply that microbial dysbiosis and translocation are the primary triggers of severe COVID-19, as the complex clinical syndrome of severe COVID-19 likely embodies multiple pathophysiological pathways. Also, our in vivo analyses do not unequivocally demonstrate a causal relationship between gut dysfunction and COVID-19 severity. However, the robust literature indicating that a disrupted intestinal barrier and microbial dysbiosis and translocation fuel inflammation and disease severity during ARDS¹¹⁻¹⁴ supports our hypothesis and is consistent with our findings.

SARS-CoV-2 infection can affect the gastrointestinal tract (GI) tract and cause GI symptoms.^{56,57} Recently, it has been suggested that the severity of GI symptoms (mainly vomiting and diarrhea) correlates inversely with COVID-19 severity (for unclear reasons).⁵⁸ On the other hand, our observations suggest that disruption in gut function and higher microbial translocation correlate positively with COVID-19 severity. These are not necessarily mutually exclusive findings, but rather indicate that the interplay between the gut and SARS-CoV-2 infection in modulating disease severity is complex.

### Example 3: Long-COVID is Associated with an Increase in Markers of Fungal Translocation

We examined both bacteria (measured as levels of LPS binding protein, LBP) and fungus (measured as levels of β-glucan) in the plasma of 117 COVID-19 patients four months after their SARS-CoV-2 qPCR negative results. The patients were divided into two groups: 56 patients with no Post-acute sequalae of COVID-19 (PASC or Long-COVID) and 61 with Long-COVID. We observed higher levels of β-glucan in the plasma of patients with Long COVID compared to non-Long-COVID (in a manner linked to the number of persistent symptoms; FIG. 18B-C), indicating elevated levels of fungal translocation during Long-COVID. Differently, the levels of LBP were not significantly higher in Long-COVID patients compared to non-Long-COVID patients (P=0.055; data not shown), indicating that fungal rather than bacterial translocation is associated with Long-COVID.

β-glucan is a biomarker of lower gut integrity and higher microbial translocation during HIV infection, and its levels correlate with inflammation, immune suppression, and the development of HIV-associated comorbidities as shown in several reports.

Beyond being a biomarker of inflammation, β-glucan can directly induce inflammation following its binding to Dectin-1 and TLR2 expressed on macrophages, monocytes, and dendritic cells. The binding between β-glucan and Dectin-1 or TLR2 activates the NF-κB pathway and induces the secretion of pro-inflammatory cytokines.

Together, these data indicate that, even following recovery from acute COVID-19, some level of fungal translocation persists in some individuals, and that this fungal translocation contributes to Long COVID by inducing chronic inflammation. Targeting gut permeability is a therapeutic option to treat Long-COVID.

Each and every patent, patent application, and publication, including websites cited herein, is hereby incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

### REFERENCES

1. Guan, W. J., et al. Clinical Characteristics of Coronavirus Disease 2019 in China. The New England journal of medicine 382, 1708-1720 (2020).
2. Gandhi, R. T., Lynch, J. B. & Del Rio, C. Mild or Moderate Covid-19. The New England journal of medicine (2020).
3. Grasselli, G., et al. Baseline Characteristics and Outcomes of 1591 Patients Infected With SARS-CoV-2 Admitted to ICUs of the Lombardy Region, Italy. Jama 323, 1574-1581 (2020).
4. Richardson, S., et al. Presenting Characteristics, Comorbidities, and Outcomes Among 5700 Patients Hospitalized With COVID-19 in the New York City Area. Jama 323, 2052-2059 (2020).
5. Chen, N., et al. Epidemiological and clinical characteristics of 99 cases of 2019 novel coronavirus pneumonia in Wuhan, China: a descriptive study. Lancet 395, 507-513 (2020).
6. Blanco-Melo, D., et al. Imbalanced Host Response to SARS-CoV-2 Drives Development of COVID-19. Cell 181, 1036-1045 e1039 (2020).
7. Chen, G., et al. Clinical and immunological features of severe and moderate coronavirus disease 2019. The Journal of clinical investigation 130, 2620-2629 (2020).
8. Mehta, P., et al. COVID-19: consider cytokine storm syndromes and immunosuppression. Lancet 395, 1033-1034 (2020).
9. Huang, C., et al. Clinical features of patients infected with 2019 novel coronavirus in Wuhan, China. Lancet 395, 497-506 (2020).
10. Risitano, A. M., et al. Complement as a target in COVID-19? Nature reviews. Immunology (2020).
11. Dumas, A., Bernard, L., Poquet, Y., Lugo-Villarino, G. & Neyrolles, O. The role of the lung microbiota and the gut-lung axis in respiratory infectious diseases. Cellular microbiology 20, e12966 (2018).
12. Frati, F., et al. The Role of the Microbiome in Asthma: The Gut(-)Lung Axis. International journal of molecular sciences 20(2018).
13. Mukherjee, S. & Hanidziar, D. More of the Gut in the Lung: How Two Microbiomes Meet in ARDS. Yale J Biol Med 91, 143-149 (2018).
14. Dickson, R. P., et al. Enrichment of the lung microbiome with gut bacteria in sepsis and the acute respiratory distress syndrome. Nat Microbiol 1, 16113 (2016).
15. Lamers, M. M., et al. SARS-CoV-2 productively infects human gut enterocytes. Science (2020).
16. Brenchley, J. M., et al. CD4+ T cell depletion during all stages of HIV disease occurs predominantly in the gastrointestinal tract. J. Exp. Med. 200, 749-759 (2004).
17. Niessen, C. M. Tight junctions/adherens junctions: basic structure and function. The Journal of investigative dermatology 127, 2525-2532 (2007).
18. Turner, J. R. Intestinal mucosal barrier function in health and disease. Nature reviews. Immunology 9, 799-809 (2009).
19. O'Toole, P. W. & Jeffery, I. B. Gut microbiota and aging. Science 350, 1214-1215 (2015).
20. Thevaranjan, N., et al. Age-Associated Microbial Dysbiosis Promotes Intestinal Permeability, Systemic Inflammation, and Macrophage Dysfunction. Cell host & microbe 21, 455-466 e454 (2017).
21. Kim, K. A., Jeong, J. J., Yoo, S. Y. & Kim, D. H. Gut microbiota lipopolysaccharide accelerates inflamm-aging in mice. BMC microbiology 16, 9 (2016).
22. Fransen, F., et al. Aged Gut Microbiota Contributes to Systemical Inflammaging after Transfer to Germ-Free Mice. Frontiers in immunology 8, 1385 (2017).
23. Zhang, C., et al. Structural modulation of gut microbiota in life-long calorie-restricted mice. Nature communications 4, 2163 (2013).
24. Klingelhoefer, L. & Reichmann, H. Pathogenesis of Parkinson disease—the gut-brain axis and environmental factors. Nat Rev Neurol 11, 625-636 (2015).
25. Fragkos, K. C. & Forbes, A. Citrulline as a marker of intestinal function and absorption in clinical settings: A systematic review and meta-analysis. United European Gastroenterol J 6, 181-191 (2018).
26. Fujisaka, S., et al. Diet, Genetics, and the Gut Microbiome Drive Dynamic Changes in Plasma Metabolites. Cell Rep 22, 3072-3086 (2018).
27. Kurilshikov, A., et al. Gut Microbial Associations to Plasma Metabolites Linked to Cardiovascular Phenotypes and Risk. Circ Res 124, 1808-1820 (2019).
28. Mayneris-Perxachs, J. & Fernandez-Real, J. M. Exploration of the microbiota and metabolites within body fluids could pinpoint novel disease mechanisms. FEBS J 287, 856-865 (2020).
29. Davaatseren, M., et al. Poly-gamma-glutamic acid attenuates angiogenesis and inflammation in experimental colitis. Mediators Inflamm 2013, 982383 (2013).
30. Villeda-Gonzalez, J. D., et al. Nicotinamide reduces inflammation and oxidative stress via the cholinergic system in fructose-induced metabolic syndrome in rats. Life Sci 250, 117585 (2020).
31. Karsten, C. M., et al. Anti-inflammatory activity of IgG1 mediated by Fc galactosylation and association of FcgammaRIIB and dectin-1. Nature medicine 18, 1401-1406 (2012).
32. Cao, Y., Rocha, E. R. & Smith, C. J. Efficient utilization of complex N-linked glycans is a selective advantage for Bacteroides fragilis in extraintestinal infections. Proceedings of the National Academy of Sciences of the United States of America 111, 12901-12906 (2014).
33. Simurina, M., et al. Glycosylation of Immunoglobulin G Associates With Clinical Features of Inflammatory Bowel Diseases. Gastroenterology 154, 1320-1333 e1310 (2018).
34. Bamba, T., Matsuda, H., Endo, M. & Fujiyama, Y. The pathogenic role of Bacteroides vulgatus in patients with ulcerative colitis. J Gastroenterol 30 Suppl 8, 45-47 (1995).
35. Huang, Y. L., Chassard, C., Hausmann, M., von Itzstein, M. & Hennet, T. Sialic acid catabolism drives intestinal inflammation and microbial dysbiosis in mice. Nature communications 6, 8141 (2015).
36. Matsuda, H., et al. Characterization of antibody responses against rectal mucosa-associated bacterial flora in patients with ulcerative colitis. J Gastroenterol Hepatol 15, 61-68 (2000).
37. Bloom, S. M., et al. Commensal Bacteroides species induce colitis in host-genotype-specific fashion in a mouse model of inflammatory bowel disease. Cell host & microbe 9, 390-403 (2011).
38. Monaghan, T. M., et al. Decreased Complexity of Serum N-glycan Structures Associates with Successful Fecal Microbiota Transplantation for Recurrent Clostridioides difficile Infection. Gastroenterology 157, 1676-1678 e1673 (2019).
39. Fasano, A. Zonulin and its regulation of intestinal barrier function: the biological door to inflammation, autoimmunity, and cancer. Physiol Rev 91, 151-175 (2011).
40. El Asmar, R., et al. Host-dependent zonulin secretion causes the impairment of the small intestine barrier function after bacterial exposure. Gastroenterology 123, 1607-1615 (2002).
41. Wood Heickman, L. K., DeBoer, M. D. & Fasano, A. Zonulin as a potential putative biomarker of risk for shared type 1 diabetes and celiac disease autoimmunity. Diabetes Metab Res Rev 36, e3309 (2020).
42. Muta, T. & Takeshige, K. Essential roles of CD14 and lipopolysaccharide-binding protein for activation of toll-like receptor (TLR)2 as well as TLR4 Reconstitution of TLR2- and TLR4-activation by distinguishable ligands in LPS preparations. Eur J Biochem 268, 4580-4589 (2001).
43. Morris, A., et al. Serum (1->3)-beta-D-glucan levels in HIV-infected individuals are associated with immunosuppression, inflammation, and cardiopulmonary function. Journal of acquired immune deficiency syndromes 61, 462-468 (2012).
44. Isnard, S., et al. Plasma Levels of C-Type Lectin REG3alpha and Gut Damage in People With Human Immunodeficiency Virus. The Journal of infectious diseases 221, 110-121 (2020).
45. Whitt, D. D. & Demoss, R. D. Effect of microflora on the free amino acid distribution in various regions of the mouse gastrointestinal tract. Appl Microbiol 30, 609-615 (1975).
46. Ma, N. & Ma, X. Dietary Amino Acids and the Gut-Microbiome-Immune Axis: Physiological Metabolism and Therapeutic Prospects. Compr Rev Food Sci F 18, 221-242 (2019).
47. Blachier, F., Mariotti, F., Huneau, J. F. & Tome, D. Effects of amino acid-derived luminal metabolites on the colonic epithelium and physiopathological consequences. Amino acids 33, 547-562 (2007).
48. Schaible, U. E. & Kaufmann, S. H. A nutritive view on the host-pathogen interplay. Trends in microbiology 13, 373-380 (2005).
49. Mellor, A. L. & Munn, D. H. IDO expression by dendritic cells: tolerance and tryptophan catabolism. Nature reviews. Immunology 4, 762-774 (2004). 50. Vujkovic-Cvijin, I., et al. Dysbiosis of the gut microbiota is associated with HIV disease progression and tryptophan catabolism. Sci Transl Med 5, 193ra191 (2013).
51. Zhao, M., et al. Modulation of the Gut Microbiota during High-Dose Glycerol Monolaurate-Mediated Amelioration of Obesity in Mice Fed a High-Fat Diet. mBio 11(2020).
52. Wahlstrom, A., Sayin, S. I., Marschall, H. U. & Backhed, F. Intestinal Crosstalk between Bile Acids and Microbiota and Its Impact on Host Metabolism. Cell Metab 24, 41-50 (2016).
53. Liang, W., et al. Alterations Of Glycerophospholipid And Fatty Acyl Metabolism In Multiple Brain Regions Of Schizophrenia Microbiota Recipient Mice. Neuropsychiatr Dis Treat 15, 3219-3229 (2019).
54. Ehehalt, R., et al. Phosphatidylcholine and lysophosphatidylcholine in intestinal mucus of ulcerative colitis patients. A quantitative approach by nanoElectrospray-tandem mass spectrometry. Scand J Gastroenterol 39, 737-742 (2004).
55. Tateno, H., Kuno, A., Itakura, Y. & Hirabayashi, J. A versatile technology for cellular glycomics using lectin microarray. Methods in enzymology 478, 181-195 (2010).
56. Mao, R., et al. Manifestations and prognosis of gastrointestinal and liver involvement in patients with COVID-19: a systematic review and meta-analysis. Lancet Gastroenterol Hepatol 5, 667-678 (2020).
57. Pan, L., et al. Clinical Characteristics of COVID-19 Patients With Digestive Symptoms in Hubei, China: A Descriptive, Cross-Sectional, Multicenter Study. Am J Gastroenterol 115, 766-773 (2020).
58. Livanos, A. E., et al. Gastrointestinal involvement attenuates COVID-19 severity and mortality. medRxiv, 2020.2009.2007.20187666 (2020).
59. Ngai, J. C., et al. The long-term impact of severe acute respiratory syndrome on pulmonary function, exercise capacity and health status. Respirology 15, 543-550 (2010).
60. Wu, Q., et al. Altered Lipid Metabolism in Recovered SARS Patients Twelve Years after Infection. Scientific reports 7, 9110 (2017).
61. Vyboh, K., Jenabian, M. A., Mehraj, V. & Routy, J. P. HIV and the gut microbiota, partners in crime: breaking the vicious cycle to unearth new therapeutic targets. Journal of immunology research 2015, 614127 (2015).
62. Vazquez-Castellanos, J. F., et al. Altered metabolism of gut microbiota contributes to chronic immune activation in HIV-infected individuals. Mucosal Immunol 8, 760-772 (2015).
63. Dinh, D. M., et al. Intestinal microbiota, microbial translocation, and systemic inflammation in chronic HIV infection. J Infect Dis 211, 19-27 (2015).
64. Knoop, K. A., McDonald, K. G., Kulkarni, D. H. & Newberry, R. D. Antibiotics promote inflammation through the translocation of native commensal colonic bacteria. Gut 65, 1100-1109 (2016).
65. Brindle, J. T., et al. Rapid and noninvasive diagnosis of the presence and severity of coronary heart disease using 1H-NMR-based metabonomics. Nat Med 8, 1439-1444 (2002).
66. Oeckl, P. & Otto, M. A Review on MS-Based Blood Biomarkers for Alzheimer's Disease. Neurol Ther 8, 113-127 (2019).
67. Hennig, R., et al. Towards personalized diagnostics via longitudinal study of the human plasma N-glycome. Biochim Biophys Acta 1860, 1728-1738 (2016).
68. Keser, T., et al. Increased plasma N-glycome complexity is associated with higher risk of type 2 diabetes. Diabetologia 60, 2352-2360 (2017).
69. Trbojevic Akmacic, I., et al. Inflammatory bowel disease associates with proinflammatory potential of the immunoglobulin G glycome. Inflamm Bowel Dis 21, 1237-1247 (2015).
70. Vuckovic, F., et al. IgG Glycome in Colorectal Cancer. Clinical cancer research: an official journal of the American Association for Cancer Research 22, 3078-3086 (2016).
71. Akinkuolie, A. O., Buring, J. E., Ridker, P. M. & Mora, S. A novel protein glycan biomarker and future cardiovascular disease events. Journal of the American Heart Association 3, e001221 (2014).
72. Doig, C. J., et al. Increased intestinal permeability is associated with the development of multiple organ dysfunction syndrome in critically ill ICU patients. American journal of respiratory and critical care medicine 158, 444-451 (1998).
73. Tripathi, A., et al. Identification of human zonulin, a physiological modulator of tight junctions, as prehaptoglobin-2. Proceedings of the National Academy of Sciences of the United States of America 106, 16799-16804 (2009).
74. Fasano, A., et al. Zonulin, a newly discovered modulator of intestinal permeability, and its expression in coeliac disease. Lancet 355, 1518-1519 (2000).
75. Sturgeon, C. & Fasano, A. Zonulin, a regulator of epithelial and endothelial barrier functions, and its involvement in chronic inflammatory diseases. Tissue Barriers 4, e1251384 (2016).
76. Arrieta, M. C., Madsen, K., Doyle, J. & Meddings, J. Reducing small intestinal permeability attenuates colitis in the IL10 gene-deficient mouse. Gut 58, 41-48 (2009).
77. Kelly, C. P., et al. Larazotide acetate in patients with coeliac disease undergoing a gluten challenge: a randomised placebo-controlled study. Alimentary pharmacology & therapeutics 37, 252-262 (2013).
78. Leffler, D. A., et al. A randomized, double-blind study of larazotide acetate to prevent the activation of celiac disease during gluten challenge. Am J Gastroenterol 107, 1554-1562 (2012).
79. Tajik, N., et al. Targeting zonulin and intestinal epithelial barrier function to prevent onset of arthritis. Nature communications 11, 1995 (2020).
80. Watts, T., et al. Role of the intestinal tight junction modulator zonulin in the pathogenesis of type I diabetes in BB diabetic-prone rats. Proceedings of the National Academy of Sciences of the United States of America 102, 2916-2921 (2005).
81. Leffler, D. A., et al. Larazotide acetate for persistent symptoms of celiac disease despite a gluten-free diet: a randomized controlled trial. Gastroenterology 148, 1311-1319 e1316 (2015).
82. Paterson, B. M., Lammers, K. M., Arrieta, M. C., Fasano, A. & Meddings, J. B. The safety, tolerance, pharmacokinetic and pharmacodynamic effects of single doses of AT-1001 in coeliac disease subjects: a proof of concept study. Alimentary pharmacology & therapeutics 26, 757-766 (2007).
83. Marquez, L., et al. Effects of haptoglobin polymorphisms and deficiency on susceptibility to inflammatory bowel disease and on severity of murine colitis. Gut 61, 528-534 (2012).
84. Papp, M., et al. Haptoglobin polymorphism: a novel genetic risk factor for celiac disease development and its clinical manifestations. Clinical chemistry 54, 697-704 (2008).
85. Costacou, T., Ferrell, R. E. & Orchard, T. J. Haptoglobin genotype: a determinant of cardiovascular complication risk in type 1 diabetes. Diabetes 57, 1702-1706 (2008).
86. Costacou, T. & Orchard, T. J. The Haptoglobin genotype predicts cardio-renal mortality in type 1 diabetes. J Diabetes Complications 30, 221-226 (2016).
87. Langlois, M. R. & Delanghe, J. R. Biological and clinical significance of haptoglobin polymorphism in humans. Clinical chemistry 42, 1589-1600 (1996).
88. Wan, C., et al. Abnormal changes of plasma acute phase proteins in schizophrenia and the relation between schizophrenia and haptoglobin (Hp) gene. Amino acids 32, 101-108 (2007).
89. Curis, E., et al. Almost all about citrulline in mammals. Amino acids 29, 177-205 (2005).
90. Peters, J. H., et al. Assessment of small bowel function in critical illness: potential role of citrulline metabolism. J Intensive Care Med 26, 105-110 (2011).
91. Kao, C. C., et al. The Microbiome, Intestinal Function, and Arginine Metabolism of Healthy Indian Women Are Different from Those of American and Jamaican Women. The Journal of nutrition 146, 706-713 (2015).
92. Batista, M. A., et al. Pretreatment with citrulline improves gut barrier after intestinal obstruction in mice. JPEN. Journal of parenteral and enteral nutrition 36, 69-76 (2012).
93. Okamoto, M., Hidaka, A., Toyama, M. & Baba, M. Galectin-3 is involved in HIV-1 expression through NF-kappaB activation and associated with Tat in latently infected cells. Virus research 260, 86-93 (2019).
94. Wang, W. H., et al. The role of galectins in virus infection—A systemic literature review. J Microbiol Immunol Infect (2019).
95. Colomb, F., et al. Galectin-9 Mediates HIV Transcription by Inducing TCR-Dependent ERK Signaling. Frontiers in immunology 10, 267 (2019).
96. Chen, Y. J., et al. Galectin-3 Enhances Avian H5N1 Influenza A Virus-Induced Pulmonary Inflammation by Promoting NLRP3 Inflammasome Activation. The American journal of pathology 188, 1031-1042 (2018).
97. Mackinnon, A. C., et al. Regulation of transforming growth factor-beta1-driven lung fibrosis by galectin-3. American journal of respiratory and critical care medicine 185, 537-546 (2012).
98. Caniglia, J. L., Guda, M. R., Asuthkar, S., Tsung, A. J. & Velpula, K. K. A potential role for Galectin-3 inhibitors in the treatment of COVID-19. PeerJ 8, e9392 (2020).
99. Caniglia, J. L., Asuthkar, S., Tsung, A. J., Guda, M. R. & Velpula, K. K. Immunopathology of galectin-3: an increasingly promising target in COVID-19. F1000Res 9, 1078 (2020).
100. Li, J., et al. The mitophagy effector FUNDC1 controls mitochondrial reprogramming and cellular plasticity in cancer cells. Science signaling 13(2020).
101. Alicea, G. M., et al. Changes in Aged Fibroblast Lipid Metabolism Induce Age-Dependent Melanoma Cell Resistance to Targeted Therapy via the Fatty Acid Transporter FATP2. Cancer Discov 10, 1282-1295 (2020).
102. Gonthier, M. P., et al. Microbial aromatic acid metabolites formed in the gut account for a major fraction of the polyphenols excreted in urine of rats fed red wine polyphenols. The Journal of nutrition 133, 461-467 (2003).
103. Marhuenda-Munoz, M., et al. Microbial Phenolic Metabolites: Which Molecules Actually Have an Effect on Human Health? Nutrients 11(2019).
104. Pellock, S. J. & Redinbo, M. R. Glucuronides in the gut: Sugar-driven symbioses between microbe and host. The Journal of biological chemistry 292, 8569-8576 (2017).
105. Monagas, M., et al. Insights into the metabolism and microbial biotransformation of dietary flavan-3-ols and the bioactivity of their metabolites. Food Funct 1, 233-253 (2010).
106. Zhitao, H., et al. Research Square (2020).
107. Coman, V. & Vodnar, D. C. Hydroxycinnamic acids and human health: recent advances. J Sci Food Agric 100, 483-499 (2020).
108. Nicholson, J. K., et al. Host-Gut Microbiota Metabolic Interactions. Science 336, 1262-1267 (2012).
109. Pallares-Mendez, R., Aguilar-Salinas, C. A., Cruz-Bautista, I. & del Bosque-Plata, L. Metabolomics in diabetes, a review. Annals of Medicine 48, 89-102 (2016).
110. Skrzypecki, J., Nieweglowska, K. & Samborowska, E. Valeric Acid, a Gut Microbiota Product, Penetrates to the Eye and Lowers Intraocular Pressure in Rats. Nutrients 12(2020).
111. Selkrig, J., Wong, P., Zhang, X. & Pettersson, S. Metabolic tinkering by the gut microbiome: Implications for brain development and function. Gut Microbes 5, 369-380 (2014).
112. Farowski, F., et al. Assessment of urinary 3-indoxyl sulfate as a marker for gut microbiota diversity and abundance of Clostridiales. Gut Microbes 10, 133-141 (2019).
113. Huang, Y. Y., Martinez-Del Campo, A. & Balskus, E. P. Anaerobic 4-hydroxyproline utilization: Discovery of a new glycyl radical enzyme in the human gut microbiome uncovers a widespread microbial metabolic activity. Gut Microbes 9, 437-451 (2018).
114. Rea, K., Dinan, T. G. & Cryan, J. F. The microbiome: A key regulator of stress and neuroinflammation. Neurobiol Stress 4, 23-33 (2016).
115. Pan, L., et al. Abnormal metabolism of gut microbiota reveals the possible molecular mechanism of nephropathy induced by hyperuricemia. Acta Pharm Sin B 10, 249-261 (2020).
116. Molinero, N., Ruiz, L., Sanchez, B., Margolies, A. & Delgado, S. Intestinal Bacteria Interplay With Bile and Cholesterol Metabolism: Implications on Host Physiology. Front Physiol 10, 185 (2019).
117. Arias, N., et al. The Relationship between Choline Bioavailability from Diet, Intestinal Microbiota Composition, and Its Modulation of Human Diseases. Nutrients 12(2020).
118. Maekawa, M., et al. Butyrate and propionate production from D-mannitol in the large intestine of pig and rat. Microbial Ecology in Health and Disease 17, 169-176 (2005).
119. Jain, A., Li, X. H. & Chen, W. N. An untargeted fecal and urine metabolomics analysis of the interplay between the gut microbiome, diet and human metabolism in Indian and Chinese adults. Scientific reports 9, 9191 (2019).
120. Utzschneider, K. M., Kratz, M., Damman, C. J. & Hullar, M. Mechanisms Linking the Gut Microbiome and Glucose Metabolism. J Clin Endocrinol Metab 101, 1445-1454 (2016).
121. Mathewson, N. D., et al. Gut microbiome-derived metabolites modulate intestinal epithelial cell damage and mitigate graft-versus-host disease. Nat Immunol 17, 505-513 (2016).
122. Ridlon, J. M., Kang, D. J., Hylemon, P. B. & Bajaj, J. S. Bile acids and the gut microbiome. Curr Opin Gastroenterol 30, 332-338 (2014).
123. Xu, M., et al. Deoxycholic Acid-Induced Gut Dysbiosis Disrupts Bile Acid Enterohepatic Circulation and Promotes Intestinal Inflammation. Dig Dis Sci (2020).
124. Di Rienzi, S. C. & Britton, R. A. Adaptation of the Gut Microbiota to Modern Dietary Sugars and Sweeteners. Adv Nutr 11, 616-629 (2020).
125. Craft, I. L., Geddes, D., Hyde, C. W., Wise, I. J. & Matthews, D. M. Absorption and malabsorption of glycine and glycine peptides in man. Gut 9, 425-437 (1968).
126. Koistinen, V. M., et al. Contribution of gut microbiota to metabolism of dietary glycine betaine in mice and in vitro colonic fermentation. Microbiome 7, 103 (2019).
127. Yasumoto, K. & Sugiyama, K. Perturbation by Bestatin of Glycyl-1-leucine Absorption in Isolated Epithelial Cells from Rat Intestine. Agricultural and Biological Chemistry 44, 1339-1344 (1980).
128. Gao, J., et al. Impact of the Gut Microbiota on Intestinal Immunity Mediated by Tryptophan Metabolism. Front Cell Infect Microbiol 8, 13 (2018).
129. Krautkramer, K. A., Fan, J. & Backhed, F. Gut microbial metabolites as multi-kingdom intermediates. Nat Rev Microbiol (2020).
130. Luo, L., et al. Association between metabolic profile and microbiomic changes in rats with functional dyspepsia. RSC Advances 8, 20166-20181 (2018).
131. Zhang, L., et al. Metabolic phenotypes and the gut microbiota in response to dietary resistant starch type 2 in normal-weight subjects: a randomized crossover trial. Scientific reports 9, 4736 (2019).
132. Oliphant, K. & Allen-Vercoe, E. Macronutrient metabolism by the human gut microbiome: major fermentation by-products and their impact on host health. Microbiome 7, 91 (2019).
133. Kong, J.-Q. Phenylalanine ammonia-lyase, a key component used for phenylpropanoids production by metabolic engineering. RSC Advances 5, 62587-62603 (2015).
134. Heianza, Y., et al. Long-Term Changes in Gut Microbial Metabolite Trimethylamine N-Oxide and Coronary Heart Disease Risk. Journal of the American College of Cardiology 75, 763-772 (2020).
135. Morris, A., Hillenbrand, M., Finkelman, M., George, M. P., Singh, V., Kessinger, C., Lucht, L., Busch, M., McMahon, D., Weinman, R., Steele, C., Norris, K. A. & Gingo, M. R. Serum (1->3)-beta-D-glucan levels in HIV-infected individuals are associated with immunosuppression, inflammation, and cardiopulmonary function. J Acquir Immune Defic Syndr 61, 462-468, doi:10.1097/QAI.0b013e318271799b (2012), PMID:22972021, PMC3494803.
136. Mehraj, V., Ramendra, R., Isnard, S., Dupuy, F. P., Ponte, R., Chen, J., Kema, I., Jenabian, M. A., Costinuik, C. T., Lebouche, B., Thomas, R., Cote, P., Leblanc, R., Baril, J. G., Durand, M., Chartrand-Lefebvre, C., Tremblay, C., Ancuta, P., Bernard, N. F., Sheppard, D. C., Routy, J. P., Montreal Primary, H. I. V. I. S., Canadian, H. I. V. & Aging Cohort Study, G. Circulating (1->3)-beta-D-glucan Is Associated With Immune Activation During Human Immunodeficiency Virus Infection. Clin Infect Dis 70, 232-241, doi:10.1093/cid/ciz212 (2020), PMID:30877304, PMC6938980.
137. Dirajlal-Fargo, S., Moser, C., Rodriguez, K., El-Kamari, V., Funderburg, N. T., Bowman, E., Brown, T. T., Hunt, P. W., Currier, J. & McComsey, G. A. Changes in the Fungal Marker beta-D-Glucan After Antiretroviral Therapy and Association With Adiposity. Open Forum Infect Dis 6, ofz434, doi:10.1093/ofid/ofz434 (2019), PMID:31737737, PMC6847395.
138. Hoenigl, M., Perez-Santiago, J., Nakazawa, M., de Oliveira, M. F., Zhang, Y., Finkelman, M. A., Letendre, S., Smith, D. & Gianella, S. (1->3)-beta-d-Glucan: A Biomarker for Microbial Translocation in Individuals with Acute or Early HIV Infection? Front Immunol 7, 404, doi:10.3389/fimmu.2016.00404 (2016), PMID:27752257, PMC5046804.
139. Isnard, S., Fombuena, B., Sadouni, M., Lin, J., Richard, C., Routy, B., Ouyang, J., Ramendra, R., Peng, X., Zhang, Y., Finkelman, M., Tremblay-Sher, D., Tremblay, C., Chartrand-Lefebvre, C., Durand, M. & Routy, J. P. Circulating beta-d-Glucan as a Marker of Subclinical Coronary Plaque in Antiretroviral Therapy-Treated People With Human Immunodeficiency Virus. Open Forum Infect Dis 8, ofab109, doi:10.1093/ofid/ofab109 (2021), PMID:34189152, PMC8232386.
140. Isnard, S., Lin, J., Bu, S., Fombuena, B., Royston, L. & Routy, J. P. Gut Leakage of Fungal-Related Products: Turning Up the Heat for HIV Infection. Front Immunol 12, 656414, doi:10.3389/fimmu.2021.656414 (2021), PMID:33912183, PMC8071945.
141. Ali, M. F., Driscoll, C. B., Walters, P. R., Limper, A. H. & Carmona, E. M. beta-Glucan-Activated Human B Lymphocytes Participate in Innate Immune Responses by Releasing Proinflammatory Cytokines and Stimulating Neutrophil Chemotaxis. J Immunol 195, 5318-5326, doi:10.4049/jimmunol.1500559 (2015), PMID:26519534, PMC4655155.
142. Elder, M. J., Webster, S. J., Chee, R., Williams, D. L., Hill Gaston, J. S. & Goodall, J. C. beta-Glucan Size Controls Dectin-1-Mediated Immune Responses in Human Dendritic Cells by Regulating IL-1beta Production. Front Immunol 8, 791, doi:10.3389/fimmu.2017.00791 (2017), PMID:28736555, PMC5500631.
143. McDonald, J. U., Rosas, M., Brown, G. D., Jones, S. A. & Taylor, P. R. Differential dependencies of monocytes and neutrophils on dectin-1, dectin-2 and complement for the recognition of fungal particles in inflammation. PLoS One 7, e45781, doi:10.1371/journal.pone.0045781 (2012), PMID:23049859, PMC3458947.

1. A method for detecting an increased risk of moderate or severe COVID-19 illness in a subject, the method comprising:
a) detecting the level of a subject biomarker in a sample from a subject having, or suspected of having, an illness associated with a coronavirus infection;
b) comparing the level of the subject biomarker to a control level;
c) diagnosing the subject with an increased risk of moderate or severe respiratory illness when an increase in the level of the subject biomarker is detected as compared to a control; and
d) treating the subject for moderate or severe illness when an increased risk is detected.

2. The method according to claim 1, wherein said subject biomarker is a biomarker of intestinal barrier integrity and is selected from zonulin, LPS binding protein, β-glucan, and regenerating islet-derived protein 3 alpha (REG3a).

3. The method according to claim 1, wherein said subject biomarker is a biomarker of inflammation and is selected from sCD14, myeloperoxidase (MPO), soluble CD163, IL-6, IL-1β, CRP, d-dimer, galectin-3, galectin-9, C3a, and GDF-15.

4. The method according to claim 1, wherein the subject biomarkers comprise zonulin, LBP, or β-glucan and one or more marker of inflammation.

5. The method according to claim 1, wherein the subject biomarkers comprise zonulin.

6. The method according to claim 1, wherein the subject biomarkers comprise one or more of CRP, IL-1, GDF-15, d-dimer, MPO, IP-10, IL-10, GAL-9, MCP-2, IL-15, MIP-1a, GAL-3, C3a, IL-1B, IL-22, TNF-a, IL-21, and or fractalkine.

7. The method according to claim 1, wherein the subject biomarkers comprise zonulin and one or more of CRP, IL-1, GDF-d-dimer, MPO, IP-10, IL-10, GAL-9, MCP-2, IL-15, MIP-1a, GAL-3, C3a, IL-1B, IL-22, TNF-a, IL-21, fractalkine, or IFN-γ.

8. The method according to claim 1, wherein the subject biomarkers comprise LBP and one or more of CRP, IL-1, GDF-15, d-dimer, MPO, IP-10, IL-10, GAL-9, MCP-2, IL-15, MIP-1a, GAL-3, C3a, IL-1B, IL-22, TNF-α, or IFN-γ.

9. The method according to claim 1, wherein the subject biomarkers comprise β-glucan and one or more of CRP, IL-1, GDF-d-dimer, MPO, IP-10, IL-10, GAL-9, MCP-2, MIP-1a, GAL-3, C3a, IL-1B, IL-22, or TNF-α.

10. The method according to claim 1, wherein the subject biomarkers comprise zonulin, LBP and sCD14.

11. A method for detecting an increased risk of moderate or severe COVID 19 illness in a subject, the method comprising:
a) detecting the levels of metabolites in the plasma of a subject having, or suspected of having a respiratory illness associated with a coronavirus infection;
b) comparing the levels of the metabolites to control levels;
c) diagnosing the subject with a higher risk of moderate or severe respiratory illness when a significant change is detected in 10 or more metabolites selected from Table 2 in the subject's plasma as compared to a control; and
d) treating the subject for severe respiratory illness.

12. (canceled)

13. A method for detecting an increased risk of moderate or severe respiratory illness associated with coronavirus infection in a subject, the method comprising:
a) detecting the level of citrulline in the plasma of a subject having, or suspected of having a respiratory illness associated with a coronavirus infection;
b) comparing the levels of citrulline to a control level;
c) diagnosing the subject with a higher risk of moderate or severe respiratory illness when a significant decrease is detected in the citrulline level in the subject's plasma as compared to a control; and
d) treating the subject for severe respiratory illness.

14. A method for detecting an increased risk of moderate or severe respiratory illness associated with coronavirus infection in a subject, the method comprising:
a) detecting the level of succinic acid or ratio of kynurenine/tryptophan [Kyn/Trp] in the plasma of a subject having, or suspected of having a respiratory illness associated with a coronavirus infection;
b) comparing the levels of succinic acid or ratio of kynurenine/tryptophan [Kyn/Trp] to a control level;
c) diagnosing the subject with a higher risk of moderate or severe respiratory illness when a significant increase is detected in the succinic acid level or ratio of kynurenine/tryptophan [Kyn/Trp] in the subject's plasma as compared to a control; and
d) treating the subject for severe respiratory illness.

15-17. (canceled)

18. The method according to claim 1, wherein the treatment comprises oxygen therapy, remdesivir, dexamethasone (or other corticosteroid), treatment to reduce gut permeability, treatment to repair or improve gut barrier integrity, or dietary change.

19. (canceled)

20. The method according to claim 1, wherein the treatment comprises administration of a zonulin receptor antagonist or blocking the zonulin pathway.

21. The method according to claim 20, wherein the zonulin receptor antagonist is larazotide acetate, INN-202, SPD-550, INN-217, and/or INN-289.

22-24. (canceled)

25. The method according to claim 1, wherein the treatment comprises increasing the level of citrulline level in the subject.

26. The method according to claim 1, wherein the treatment comprises inhibiting or reducing the level of one or more galectins in the subject.

27. The method according to claim 26, wherein the galectin is GAL-3 or GAL-9.

28. The method according to claim 26 comprising administering a small molecule galectin inhibitor.

29-32. (canceled)


SEQUENCE LISTING (20 sequences)

SEQ ID NO: 1 - protein, 346 aa, Homo sapiens
MRALGAVVTLLLWGQLFAVDLSNDAMDTADDSCPKPPEIENGYVEHLVRYRCQHYRLRTEGDGVYTLNSEKQWVNTAAGERLPECEAVCGKPKHPVDQVQRIIGGSLDAKGSFPWQAKMVSRHELITGATLISDQWLLTTAKNLFLNHSEDATSKDIAPTLKLYVGKMQPVEIEKVVIHPNRSVVDIGVIKLRQKVPVNERVMPICLPSKDYIAPGRMGYVSGWGRNANFRFTDRLKYVMLPVADQDSCMLHYEGSTVPEKEGSKSSVGVQPILNEHTFCAGMTKYQEDTCYGDAGSAFAIHDLEQDTWYAAGILSFDKSCSVAEYGVYVKVNSFLDWIQETMAKN

SEQ ID NO: 2 - protein, 481 aa, Homo sapiens
MGALARALPSILLALLLTSTPEALGANPGLVARITDKGLQYAAQEGLLALQSELLRITLPDFTGDLRIPHVGRGRYEFHSLNIHSCELLHSALRPVPGQGLSLSISDSSIRVQGRWKVRKSFFKLQGSFDVSVKGISISVNLLLGSESSGRPTVTASSCSSDIADVEVDMSGDLGWLLNLFHNQIESKFQKVLESRICEMIQKSVSSDLQPYLQTLPVTTEIDSFADIDYSLVEAPRATAQMLEVMFKGEIFHRNHRSPVTLLAAVMSLPEEHNKMVYFAISDYVFNTASLVYHEEGYLNFSITDDMIPPDSNIRLTTKSFRPFVPRLARLYPNMNLELQGSVPSAPLLNFSPGNLSVDPYMEIDAFVLLPSSSKEPVFRLSVATNVSATLTFNTSKITGFLKPGKVKVELKESKVGLFNAELLEALLNYYILNTFYPKFNDKLAEGFPLPLLKRVQLYDLGLQIHKDFLFLGANVQYMRV

SEQ ID NO: 3 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 4 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 5 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 6 - protein, 1156 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 7 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 8 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 9 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 10 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 11 - protein, 250 aa, Homo sapiens [sequence omitted]

SEQ ID NO: 12 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 13 - protein, 100 aa, Homo sapiens
  misc_feature (1)..(1): Xaa can be any naturally occurring amino acid
XFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPK

SEQ ID NO: 14 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 15 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 16 - protein, 99 aa, Homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 17 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 18 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 19 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 20 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL